(19) United States
(12) Patent Application Publication
(10) Pub. No.: US 2025/0388567 A1
(43) Pub. Date: Dec. 25, 2025

(54) SELECTIVE G PROTEIN-COUPLED RECEPTOR KINASE 5 INHIBITORS, COMPOSITIONS, AND METHODS OF USE

(71) Applicant: Purdue Research Foundation, West Lafayette, IN (US)

(72) Inventors: Arun K. GHOSH, West Lafayette, IN (US); John Tesmer, West Lafayette, IN (US); Amol Sonawane, Nashik, Maharashtra (IN); Rajesh Manda, West Lafayette, IN (US)

(21) Appl. No.: 18/843,111

(22) PCT Filed: Mar. 1, 2023
(86) PCT No.: PCT/US2023/063451

§ 371 Date: Aug. 30, 2024

Related U.S. Application Data
(60) Provisional application No. 63/315,118, filed on Mar. 1, 2022.

(51) Int. Cl. C07D 403/14; A61K 31/404; A61K 31/4184; A61K 31/423; A61K 31/427; A61K 31/437; A61K 31/4439; A61K 31/454; A61K 31/4709; A61K 31/496; A61K 31/5377; A61K 31/5395; C07D 401/14; C07D 403/06; C07D 405/14; C07D 409/14; C07D 413/14; C07D 417/14; C07D 471/04; C07D 498/04

(52) U.S. Cl. CPC C07D 403/14; A61K 31/404; A61K 31/4184; A61K 31/423; A61K 31/427; A61K 31/437; A61K 31/4439; A61K 31/454; A61K 31/4709; A61K 31/496; A61K 31/5377; A61K 31/5395; C07D 401/14; C07D 403/06; C07D 405/14; C07D 409/14; C07D 413/14; C07D 417/14; C07D 471/04; C07D 498/04

(57) ABSTRACT

Highly selective and potent inhibitors of G protein-coupled receptor kinase 5 (GRK5), a pharmaceutical composition comprising same, and a method of use, such as in the treatment of heart disease or cancer.

### CROSS-REFERENCE TO RELATED APPLICATIONS

This application is a U.S. national stage filing under 35 U.S.C. § 371 from International Application No. PCT/US2023/063451, filed on Mar. 1, 2023, and published as WO 2023/168246A2 on Sep. 7, 2023, which claims the benefit of U.S. Provisional Appl. No. 63/315,118, which was filed Mar. 1, 2022, and which applications and publication are hereby incorporated by reference in their entirety.

### STATEMENT OF GOVERNMENT SUPPORT

This invention was made with government support under HL071818 awarded by the National Institutes of Health. The government has certain rights in the invention.

### TECHNICAL FIELD

This disclosure relates to G protein-coupled receptor kinase 5 (GRK5) inhibitors, compositions comprising same, and methods of use, such as in the treatment of heart disease and cancer.

### BACKGROUND

There is a need for effective treatments of heart disease (e.g., hypertrophic cardiomyopathy and heart failure) and cancer. Selective and potent inhibitors of G protein-coupled receptor kinase 5 (GRK5) could address this need.

In view of the above, it is an object of the present disclosure to provide highly selective and potent GRK5 inhibitors. This and other objects and advantages, as well as inventive features, will be apparent from the description provided herein.

### SUMMARY

Provided are compounds of the formula:

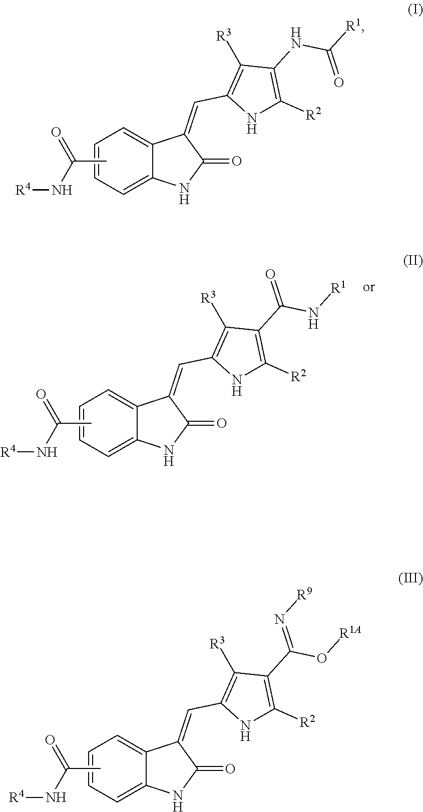

or a pharmaceutically acceptable salt thereof,
wherein:
R¹ is R⁵, -alkyl-R⁵, —C(O)R⁵,

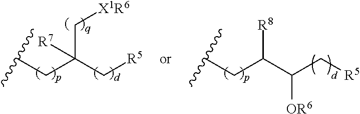

wherein R⁵ is alkyl, hydroxy, alkoxy, amido, cyano, alkenyl, haloalkyl, alkoxyalkyl, aminoalkyl, cycloalkyl, heterocyclyl, aryl or heteroaryl or R⁵ and R⁷ can form a cycloalkyl group;
R^{1A} is H or alkyl or R^{1A} and R⁹ can form a heterocyclyl group;
X¹ is O or NR^{6A}, wherein R^{6A} is H or alkyl or R^{6A} and R⁵ can form a heterocyclyl group;
p, d, and q are each, independently, an integer from 0 to 5;
R² and R³ are each, independently, alkyl;
R⁶ is H or alkyl;
R⁷ is H, alkyl or aryl;
R⁸ is H or alkyl;
R⁹ is H or alkyl; and
R⁴ is aryl, arylalkyl or heteroarylalkyl.

Such compounds are demonstrably selective and potent inhibitors of G protein-coupled receptor kinase 5 (GRK5). For example, the compounds are selective for GRK5 over GRK2. For example, the compounds can have an IC₅₀ for GRK2 of greater than 10 μM and an IC₅₀ for GRK5 of 1-999 nM (e.g., 1-100 nM).

The disclosure also relates to methods for treating a subject in need of inhibition of G protein-coupled receptor kinase 5 (GRK5), which method comprises administering to the subject in need thereof an effective amount of an above-described compound, whereupon the subject in need of inhibition of GRK5 is treated. The subject may have heart disease and/or cancer.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Provided is a compound of the formula (I), (II) or (III):

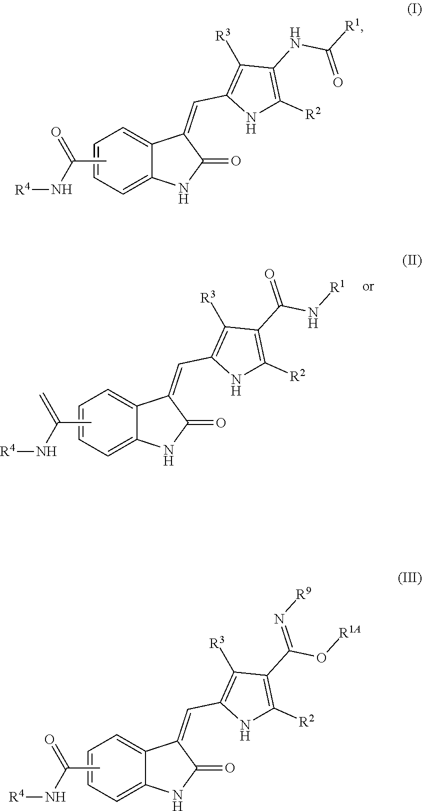

or a pharmaceutically acceptable salt thereof,
wherein:
R¹ is R⁵, -alkyl-R⁵, —C(O)R⁵,

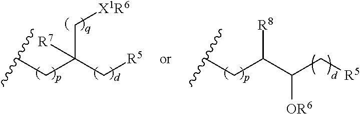

wherein R⁵ is alkyl, hydroxy, alkoxy, amido, cyano, alkenyl, haloalkyl, alkoxyalkyl, aminoalkyl, cycloalkyl, heterocyclyl, aryl or heteroaryl or R⁵ and R⁷ can form a cycloalkyl group;
R^{1A} is H or alkyl or R^{1A} and R⁹ can form a heterocyclyl group;
X¹ is O or NR^{6A}, wherein R^{6A} is H or alkyl or R^{6A} and R⁵ can form a heterocyclyl group;
p, d, and q are each, independently, an integer from 0 to 5;
R² and R³ are each, independently, alkyl;
R⁶ is H or alkyl;
R⁷ is H, alkyl or aryl;
R⁸ is H or alkyl;
R⁹ is H or alkyl; and
R⁴ is aryl, arylalkyl or heteroarylalkyl.

Examples of the compounds of the formulae (I), (II), and (III) include compounds of the formula (Ia), (IIa), and (IIIa):

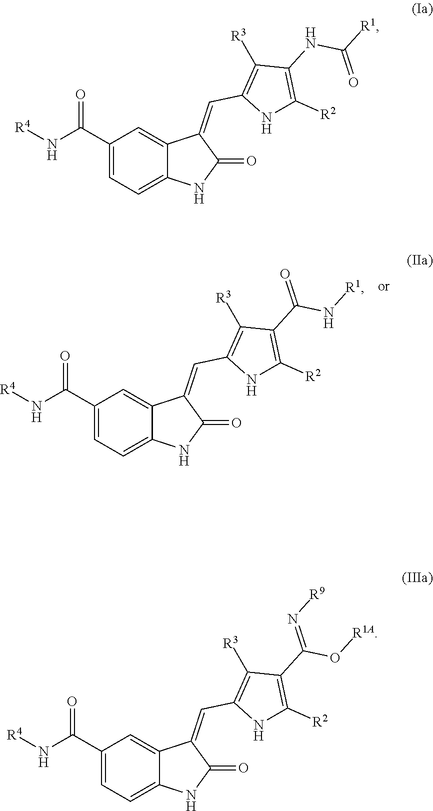

In any of the compounds (e.g, compounds of the formula (I), (Ia), (II), (IIa), (III), and (IIIa)) R² and R³ can each independently be C₁-C₃-alkyl, such as methyl. In addition or alternatively, in the compounds R⁴ can be arylalkyl, such as (C₁-C₆)alkylaryl and (C₁-C₆)alky(C₆-C₁₀)aryl. Alternatively, R⁴ can be a group of the formula (II):

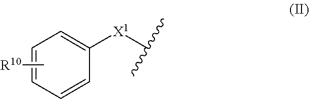

wherein R¹⁰ is H, halo, alkyl, haloalkyl, amido or alkoxy; and
X¹ is absent or alkyl (e.g., (C₁-C₆)alkylaryl and (C₁-C₆)alky(C₆-C₁₀)aryl). R¹⁰ can be halo (e.g. fluoro). Examples of groups of the formula (II) include groups of the formula:

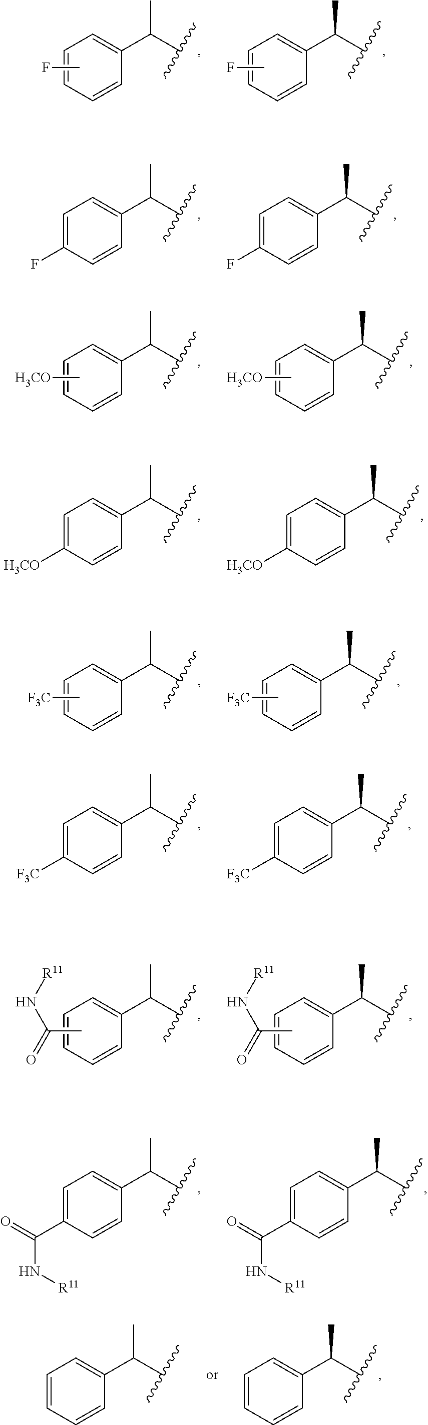

wherein R¹¹ is alkyl, which can be substituted with heterocyclyl, such as with a pyranyl group.

Examples of groups of the formula (II) also include groups of the formula:

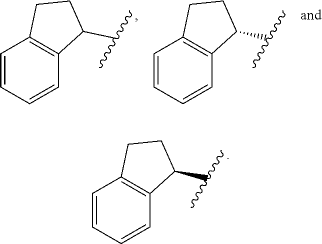

Provided is a compound of the formula (I):

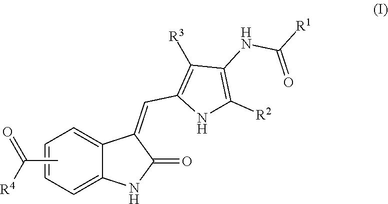

wherein R², R³, and R⁴ are defined herein and wherein R¹ is R⁵, -alkyl-R⁵, —C(O)R⁵ or

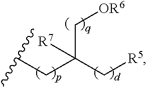

wherein R⁵ is alkyl, cycloalkyl, aryl or heteroaryl. In the compounds of the formula (I), R⁵ can be furanyl, indolyl, imidazolyl, benzimidazolyl or imidazo[1,2-a]pyridinyl]; q is 0, p is 0, and d is 0; and/or R¹ is -alkyl-R⁵, such as —(C₁-C₃)alkyl-R⁵.

Provided is a compound of the formula (II):

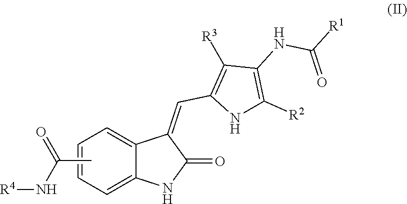

wherein R¹ is R⁵, -alkyl-R⁵ or

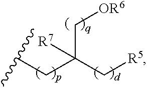

wherein R⁵ is alkyl, hydroxy, alkoxy, cycloalkyl, aryl, heterocyclyl or heteroaryl. In the compounds of the formula (II), R⁵ can be tetrahydrofuranyl, pyranyl, furanyl, tetrahydro-2H-thiopyran-1,1-dioxide or benzimidazolyl; q can be 0 and d can be 0; and/or R¹ can be -alkyl-R⁵, such as —(C₁-C₃)alkyl-R⁵. For example, in compounds of the formula (II), R¹ can be

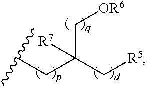

wherein R⁵ and R⁷ form a cycloalkyl group (e.g., a (C₃-C₆)cycloalkyl group). Alternatively, in the compounds of the formula (II), R¹ can be R⁵ or -alkyl-R⁵ and R⁵ can be hydroxy or alkoxy.

Examples of compounds of the formula (I), (Ia), (II), (IIa), (III), and (IIIa) include compounds of the formula:

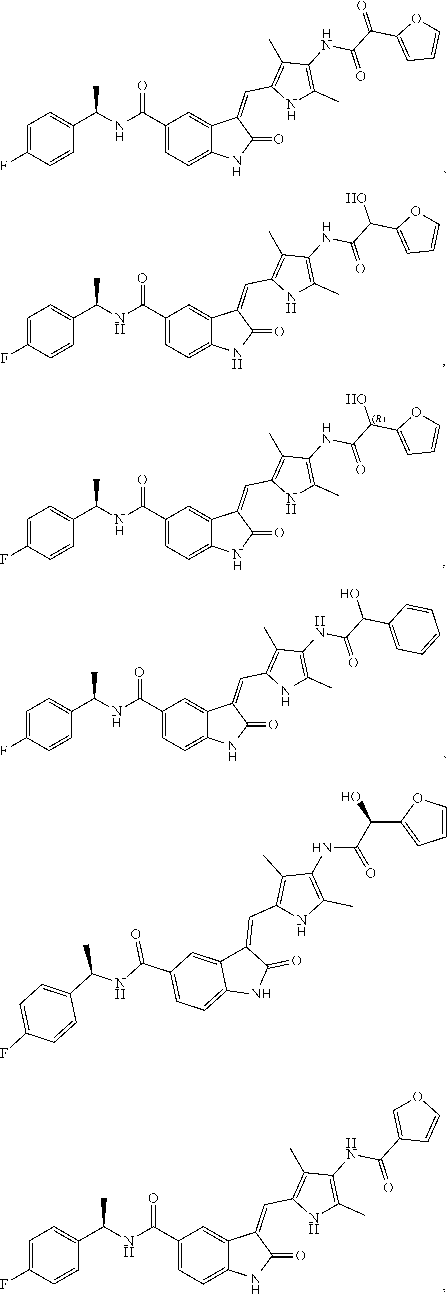

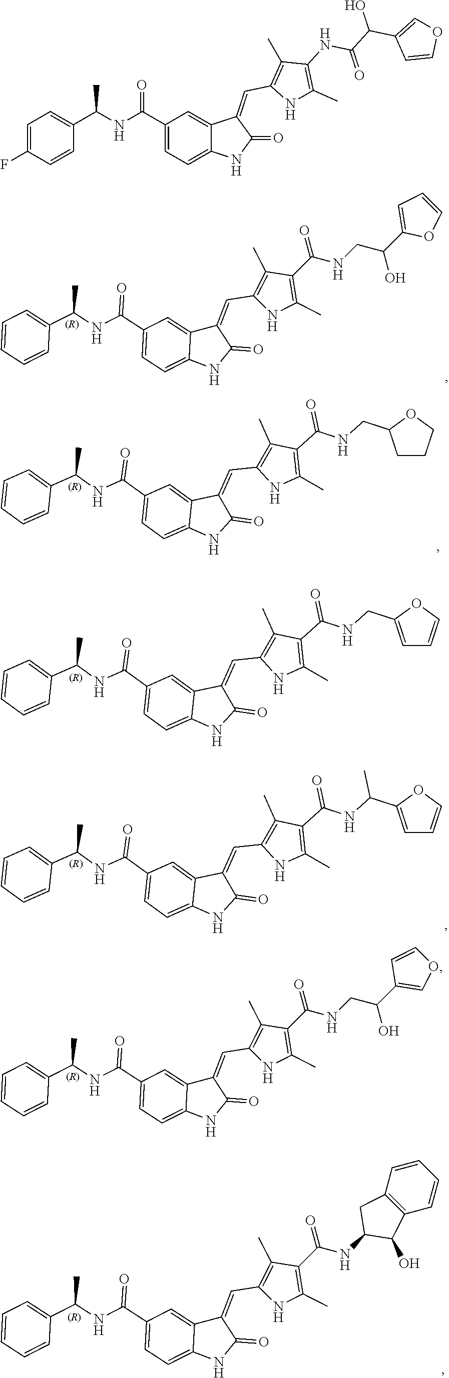

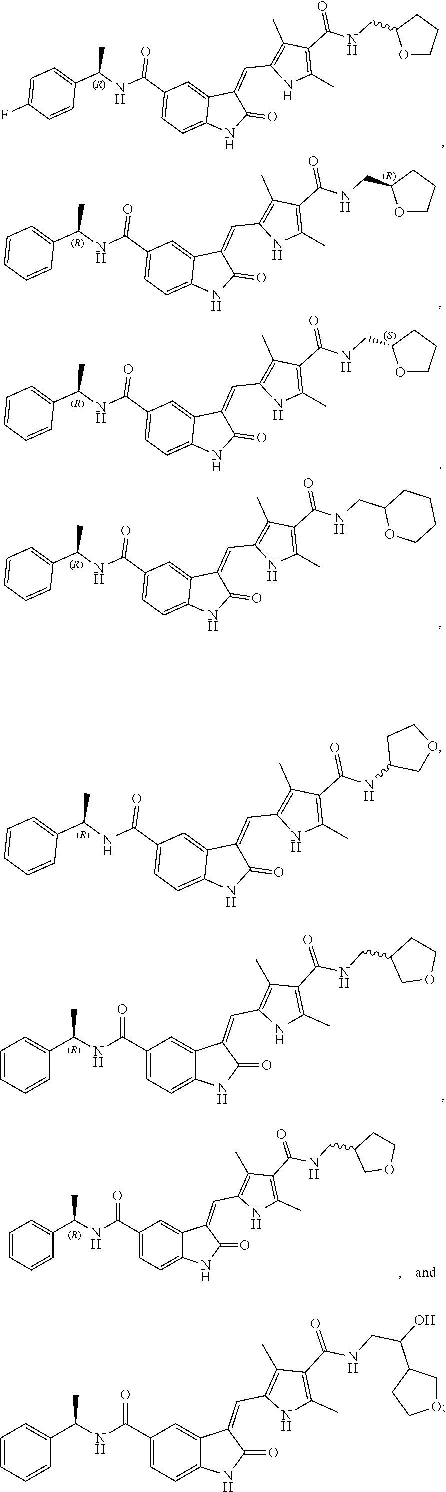

as well as compounds of the formula:

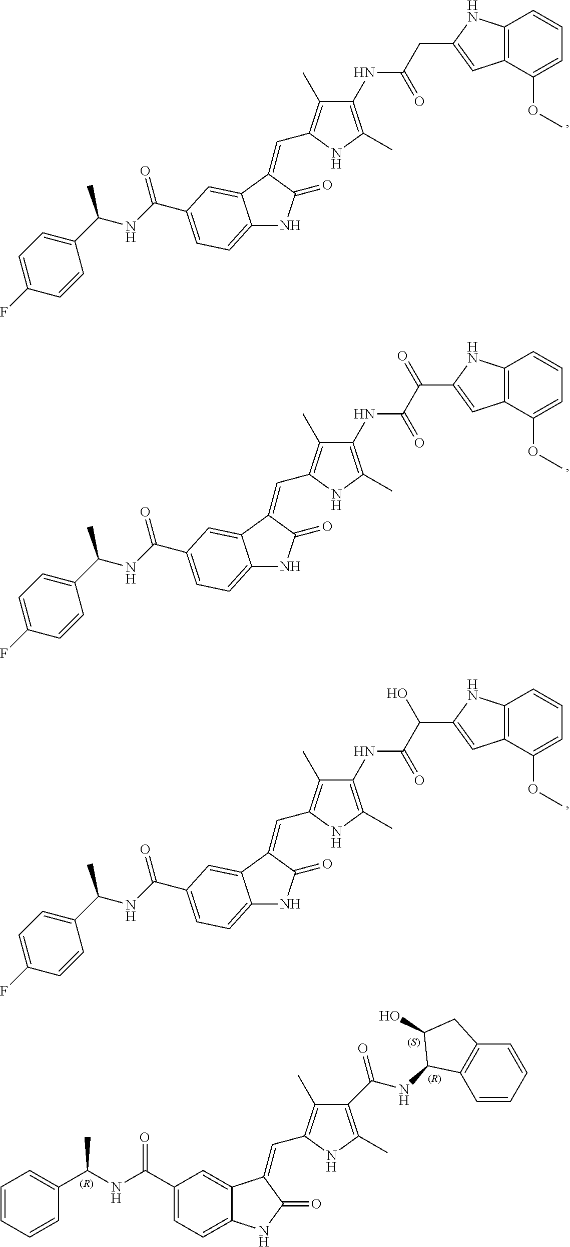

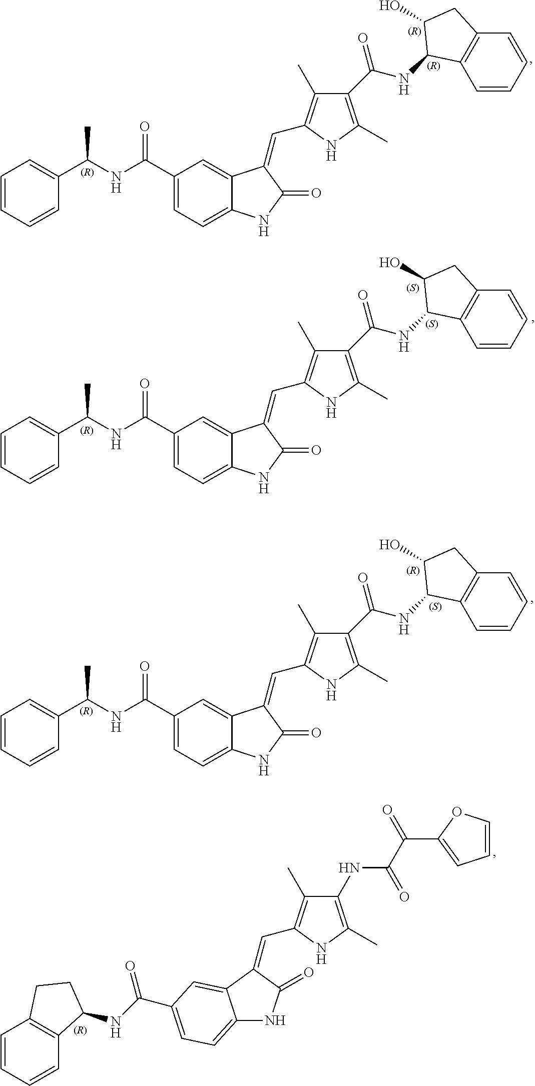

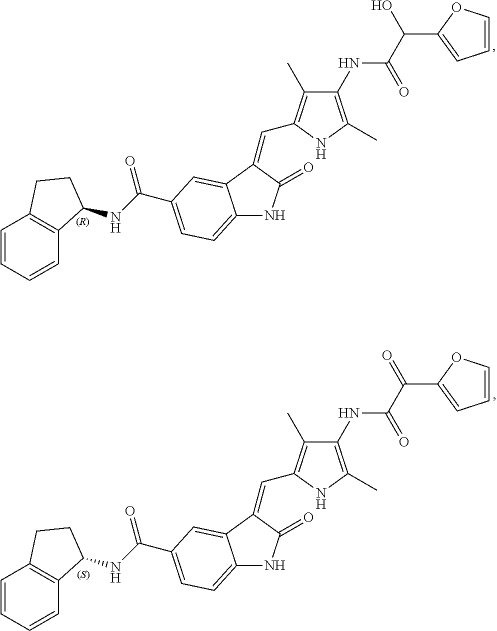

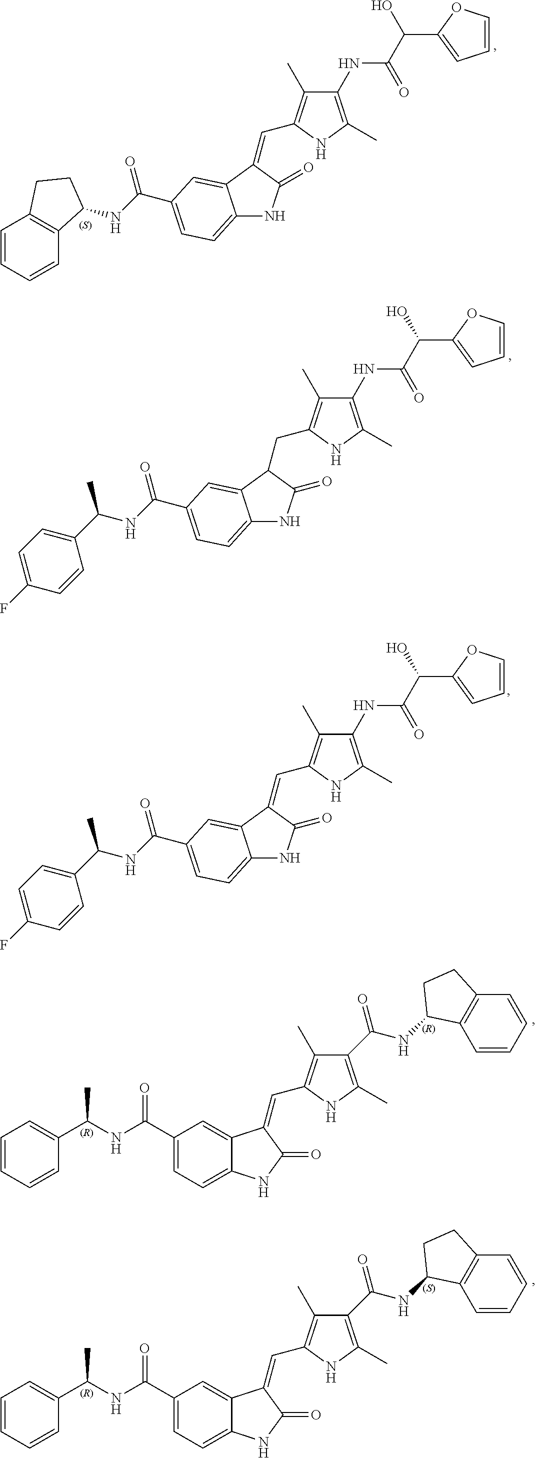

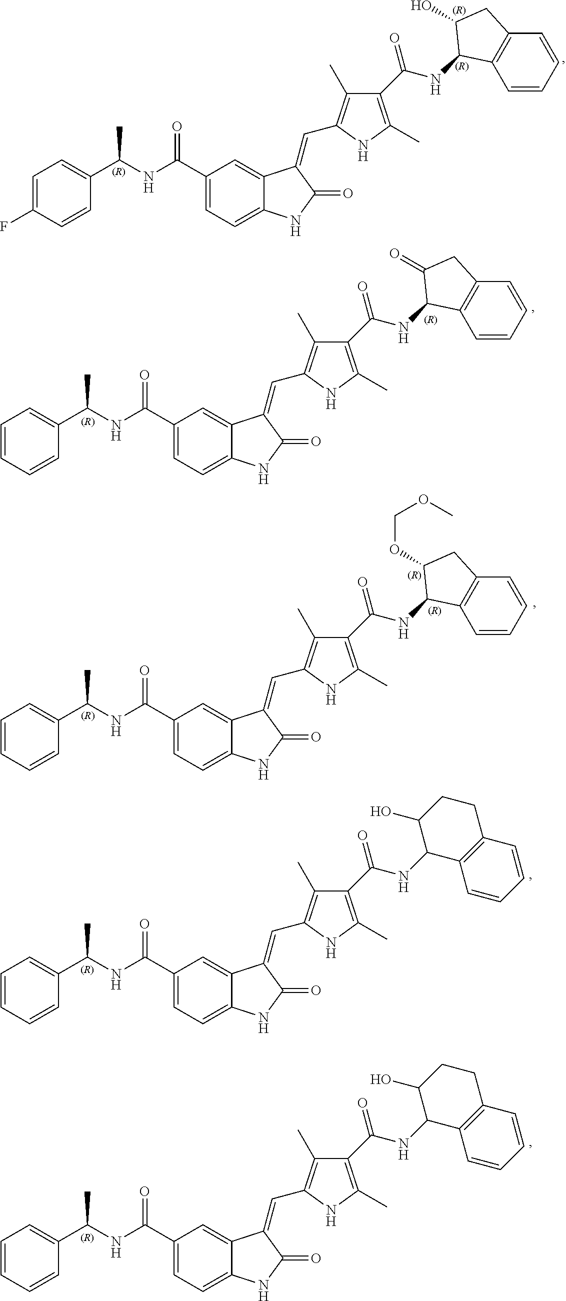

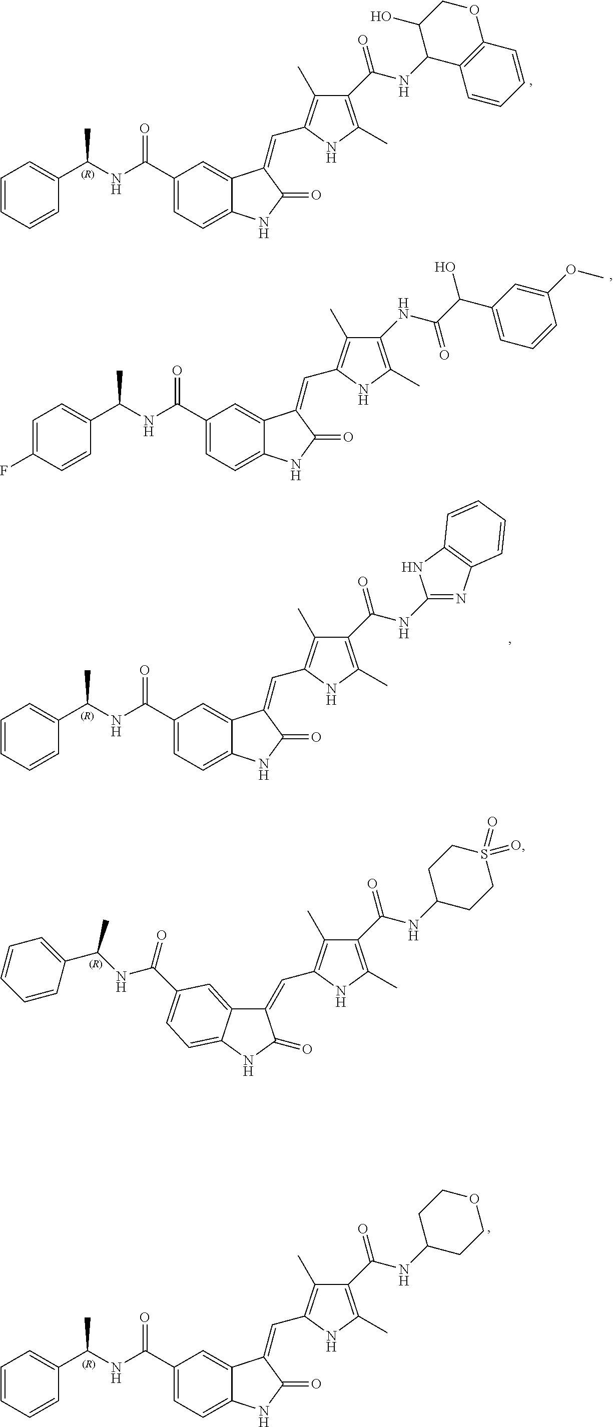

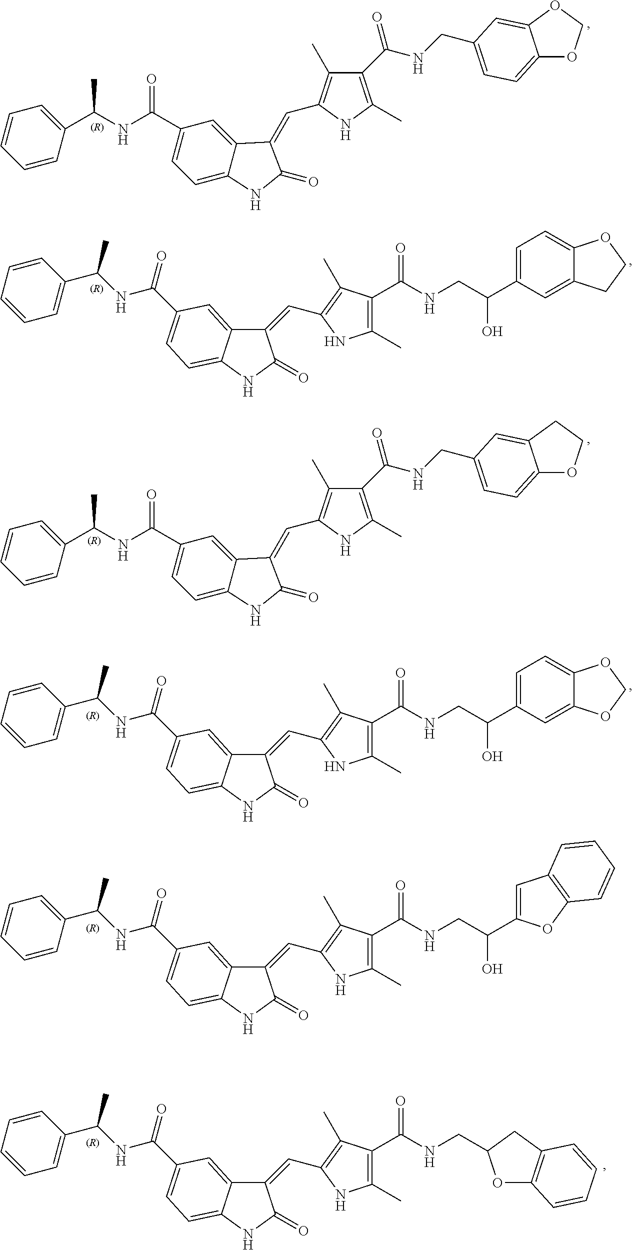

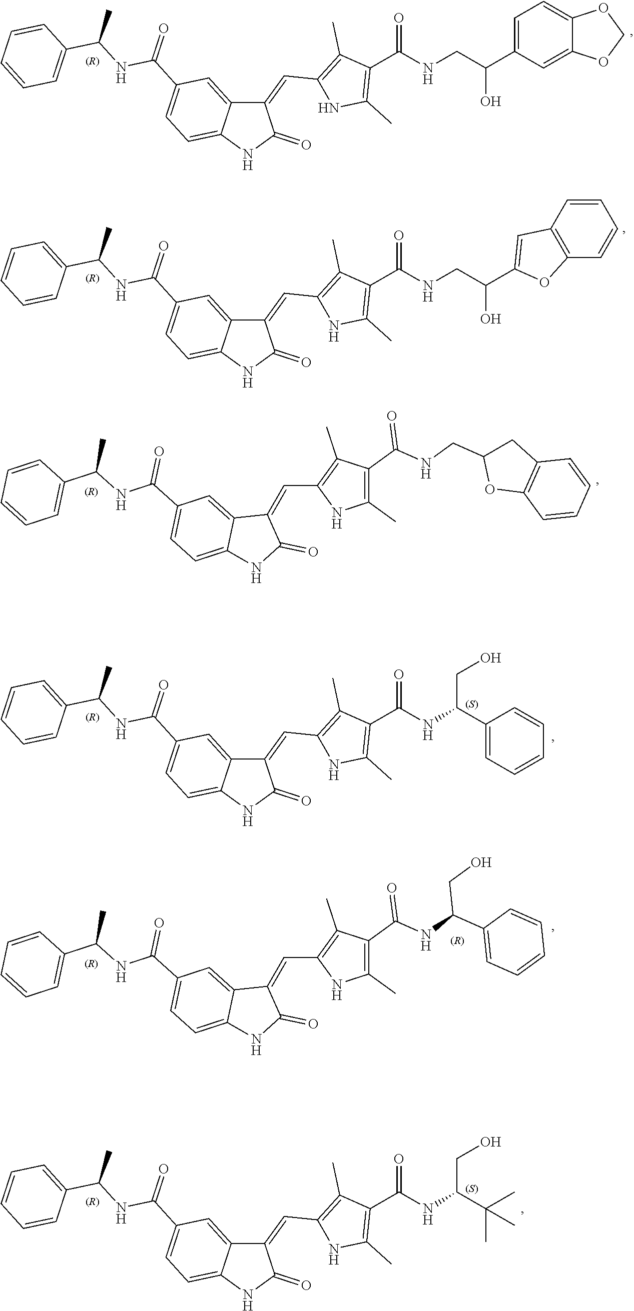

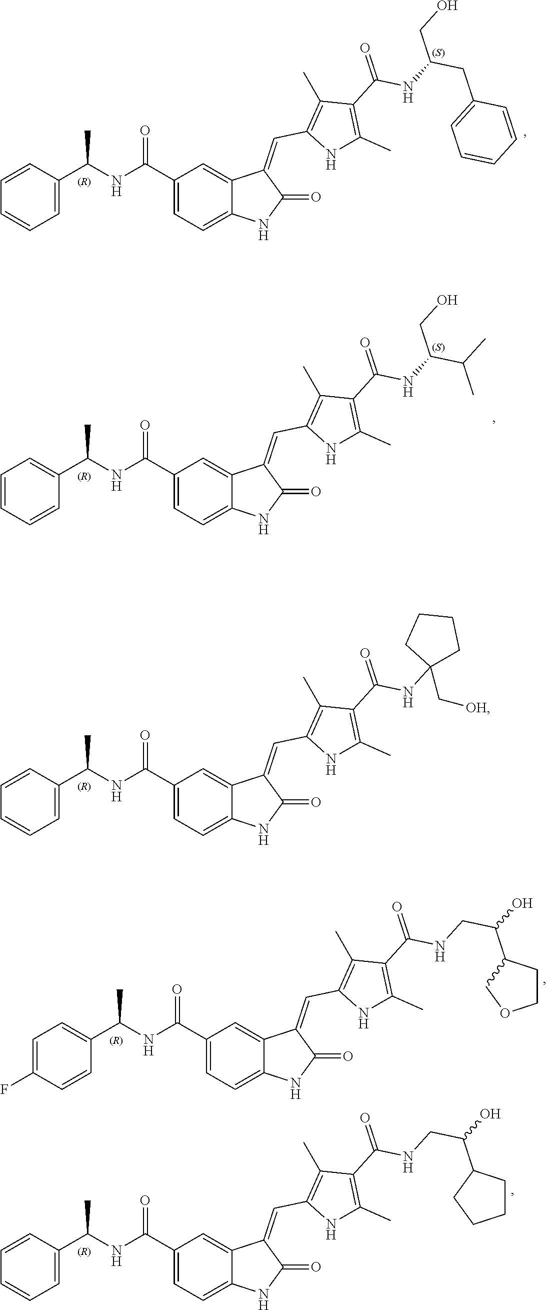

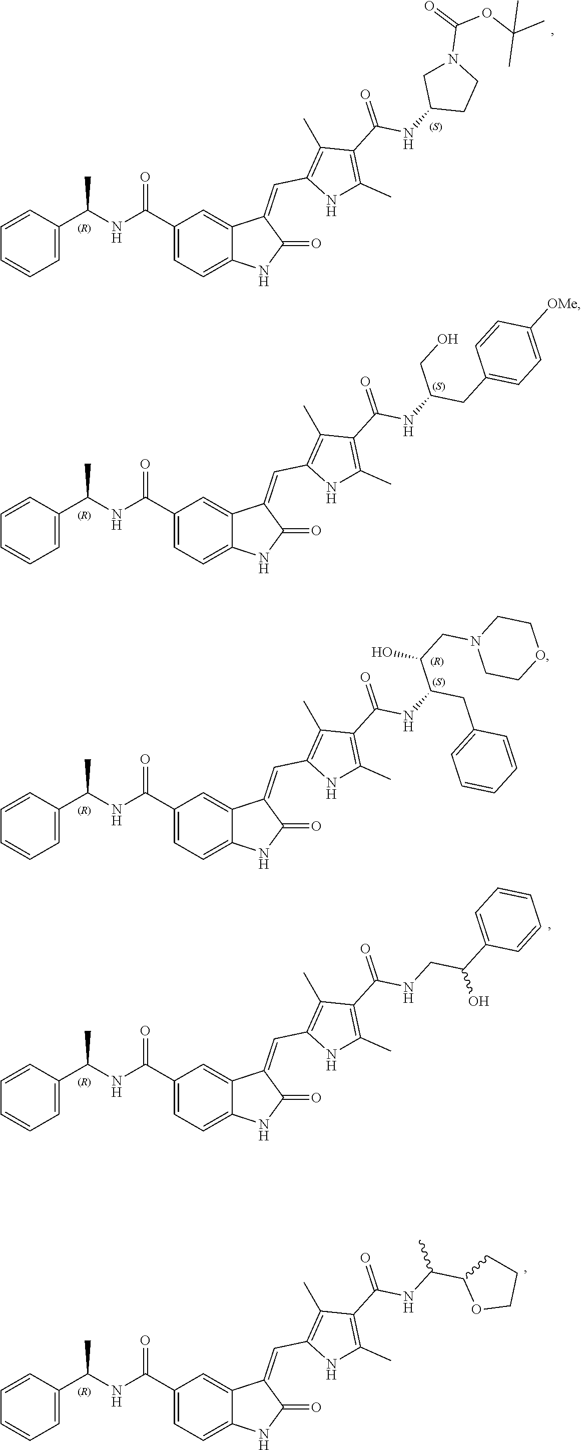

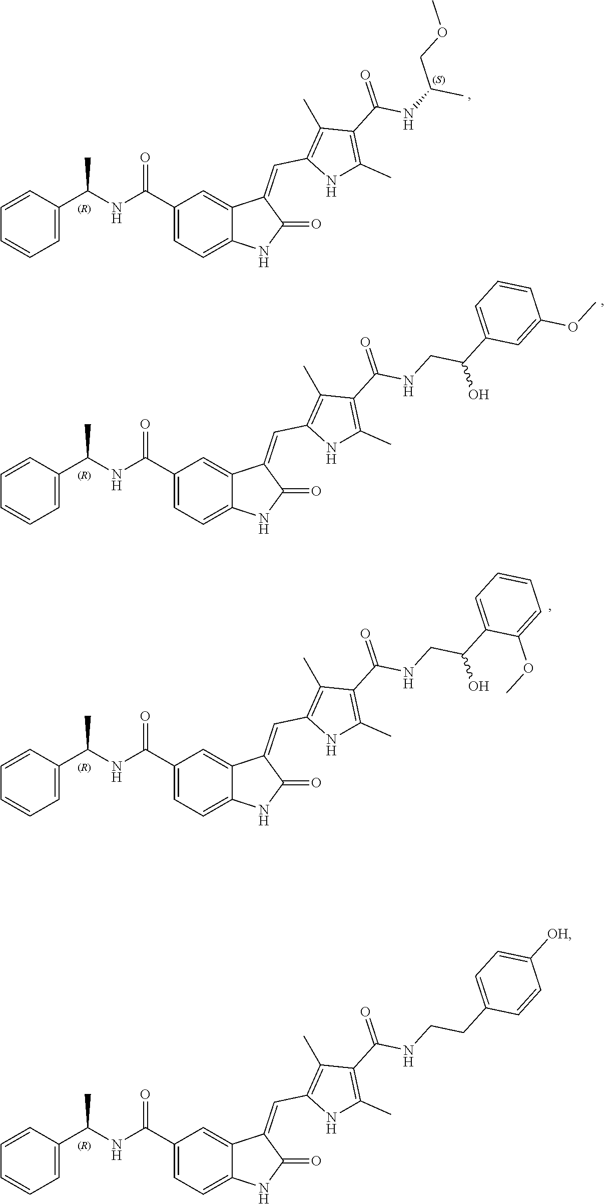

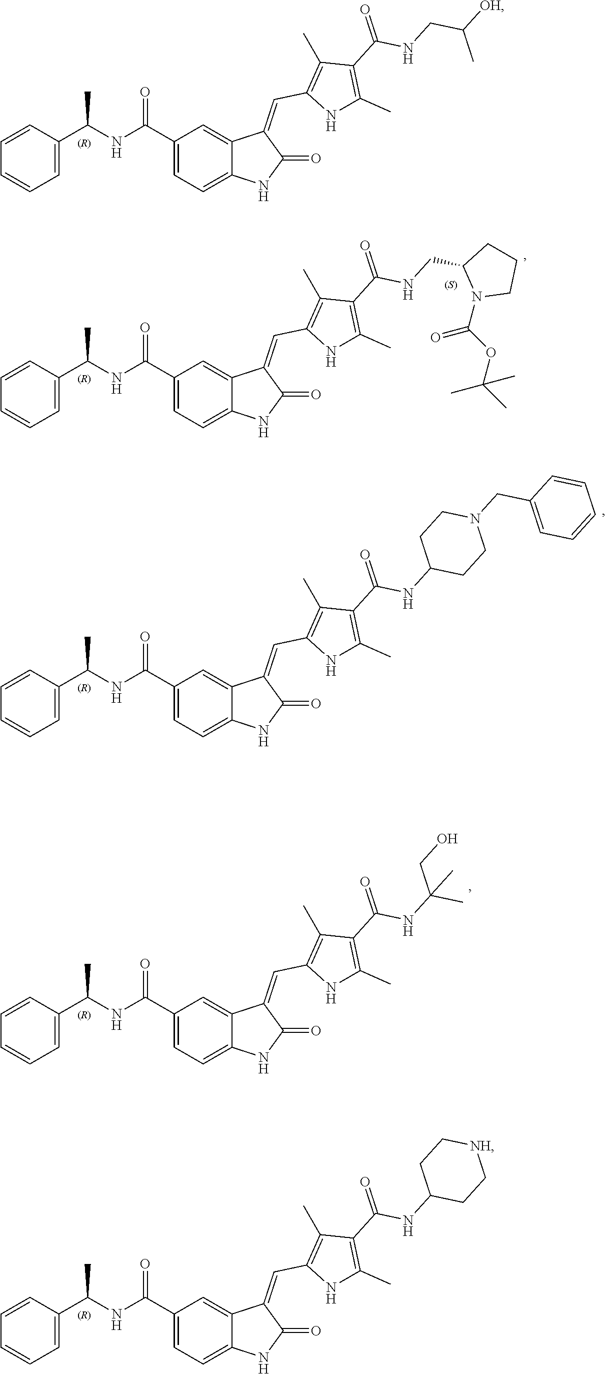

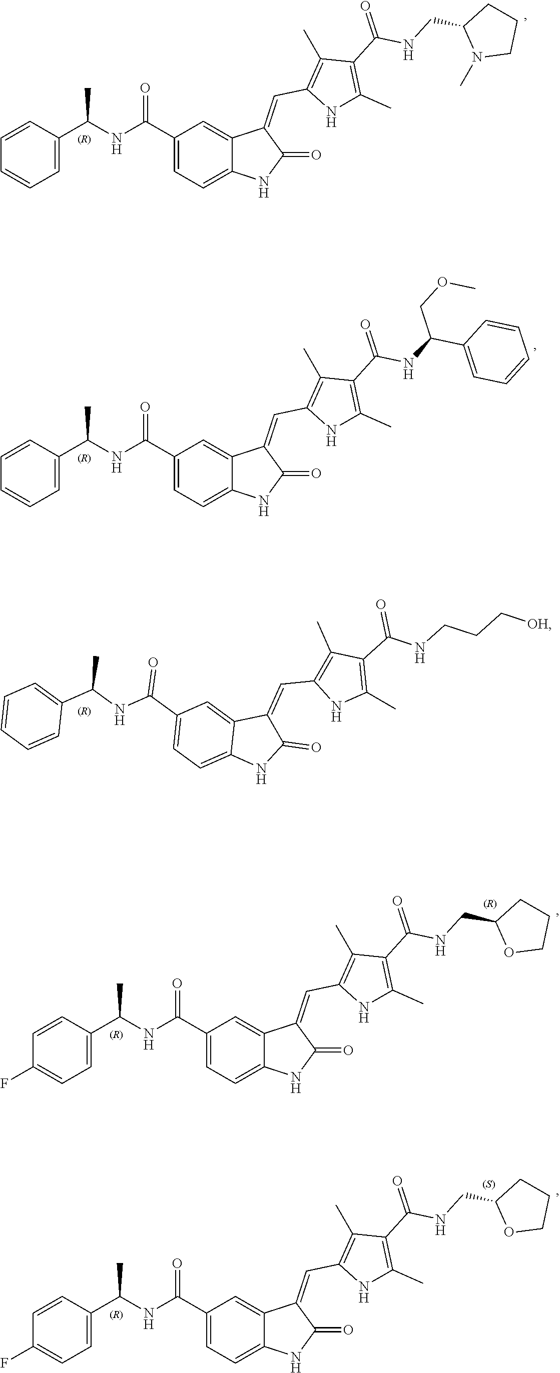

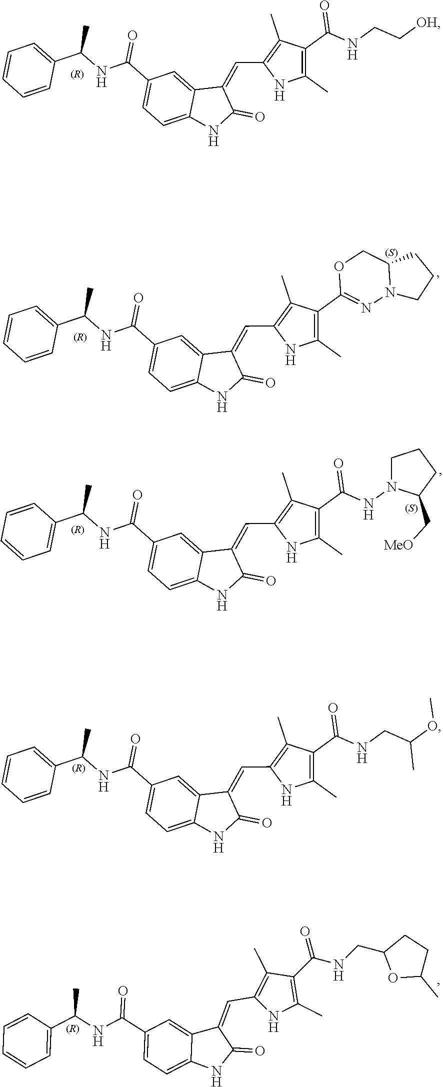

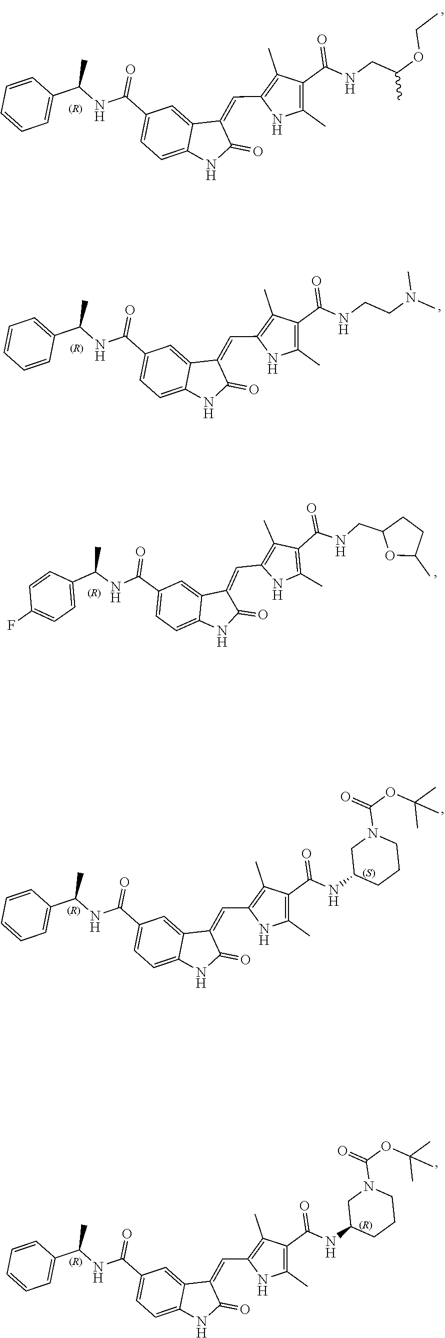

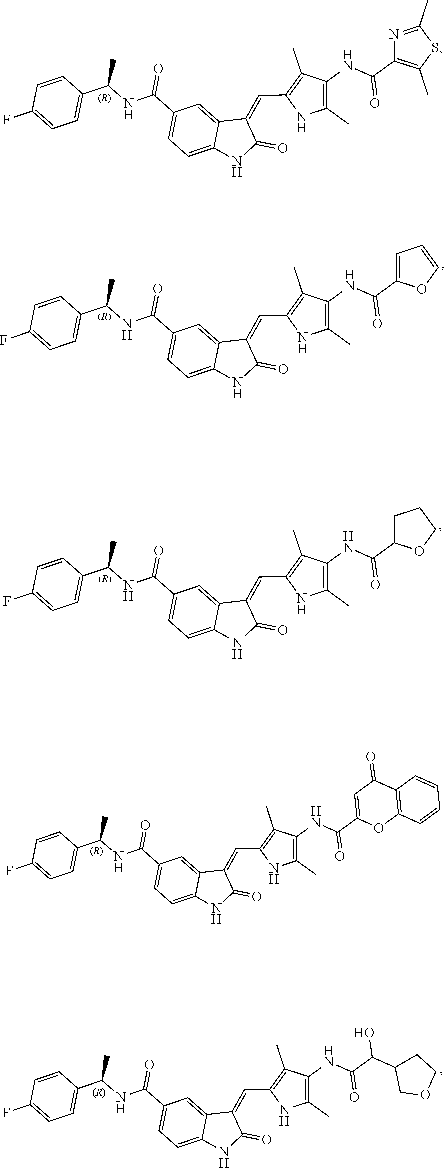

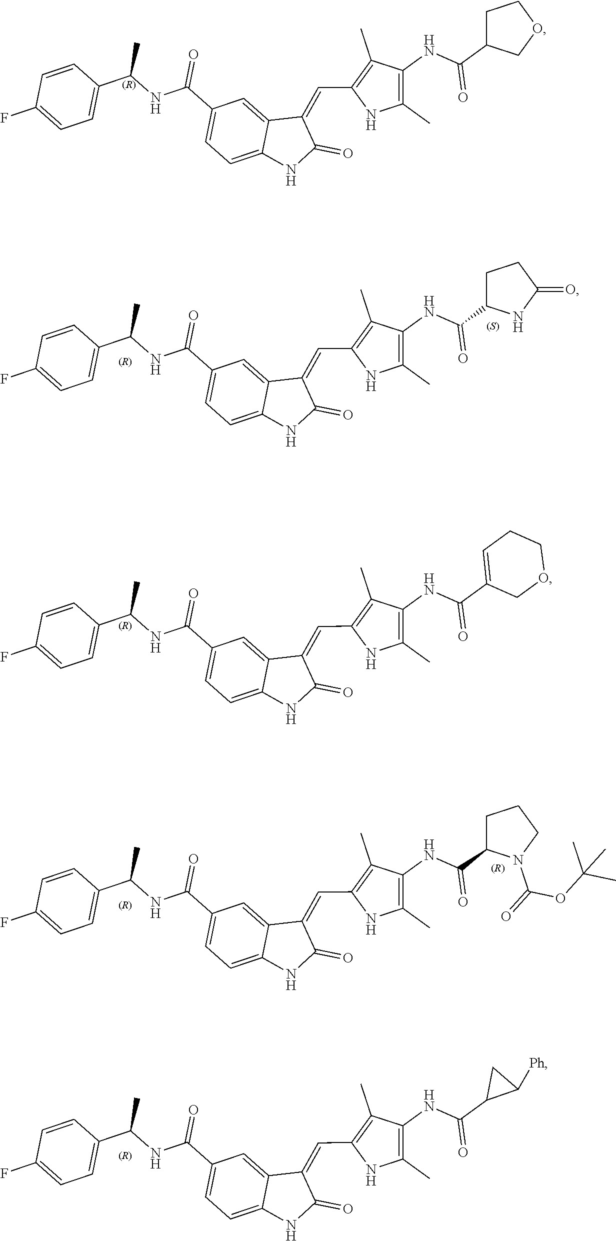

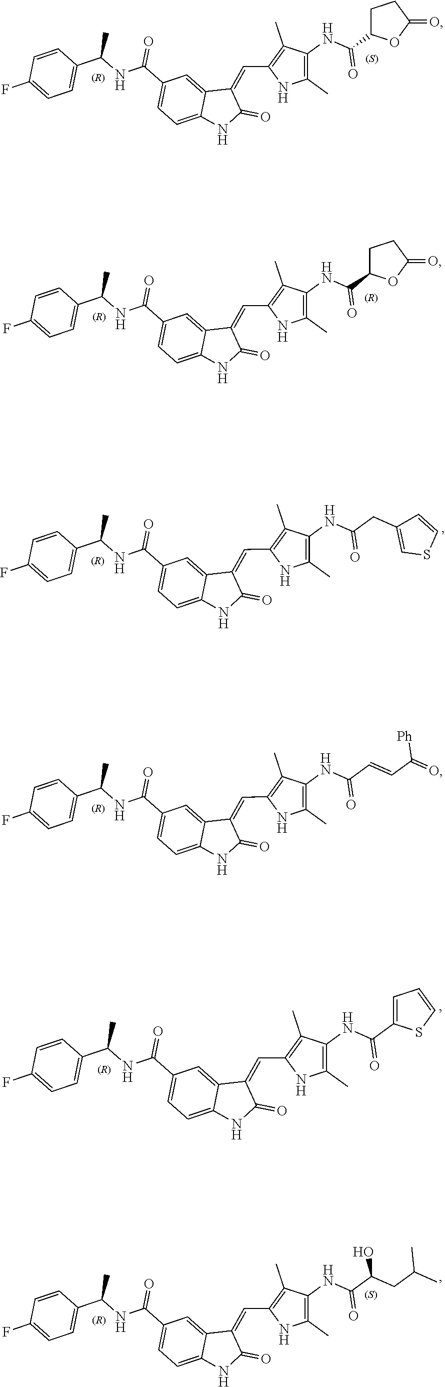

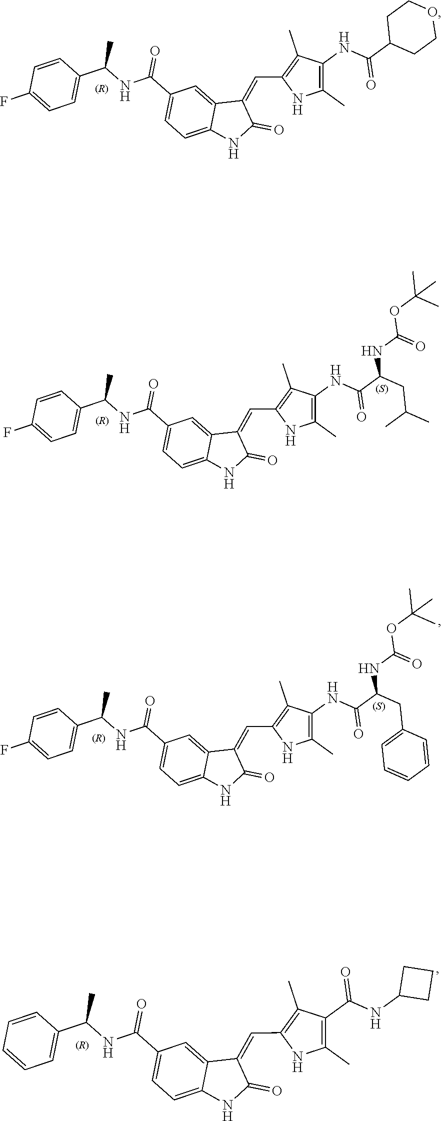

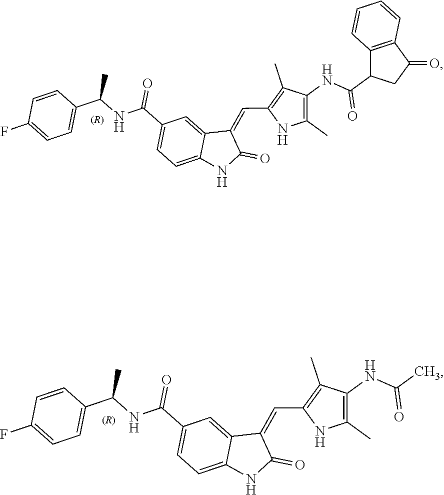

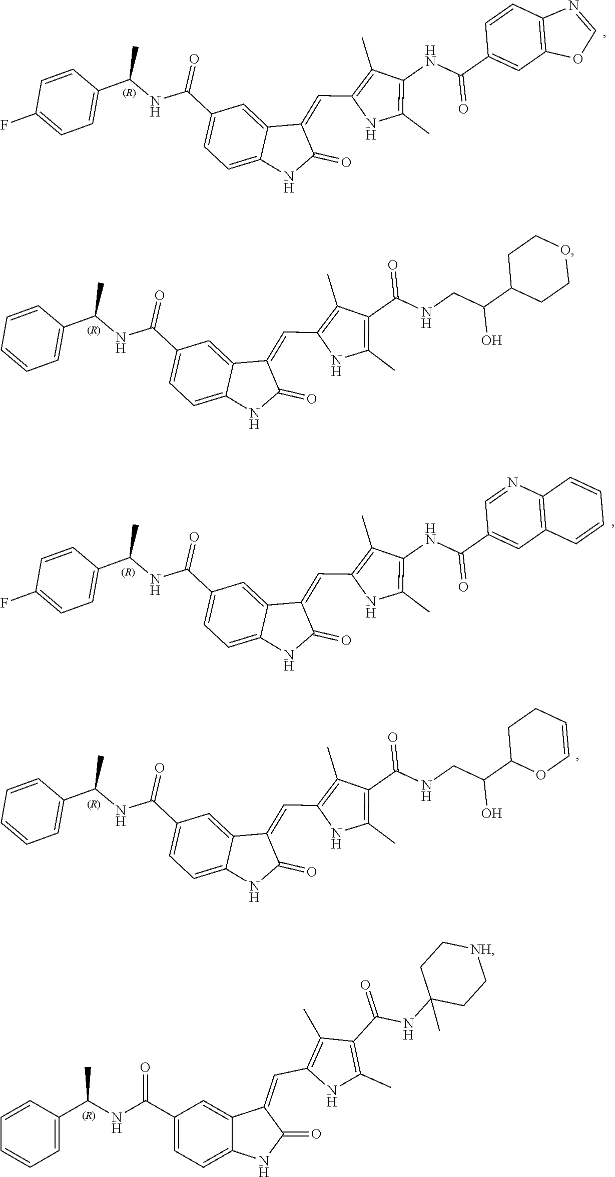

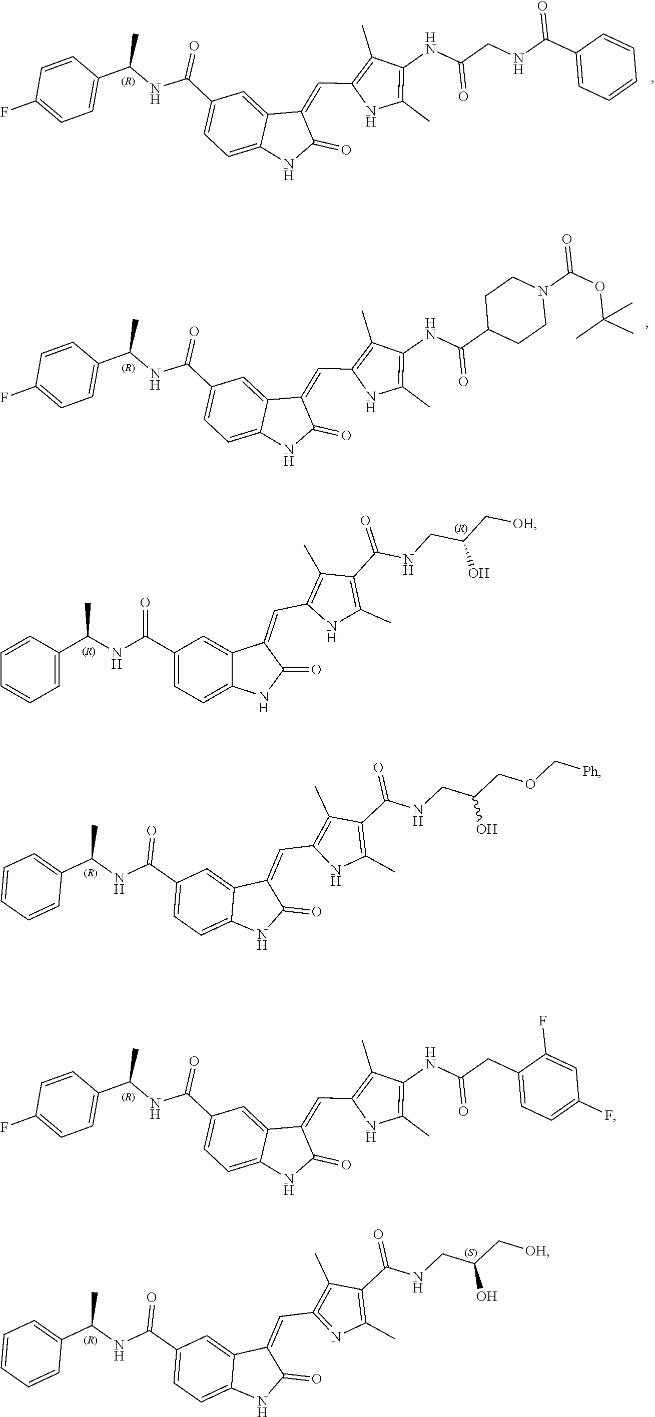

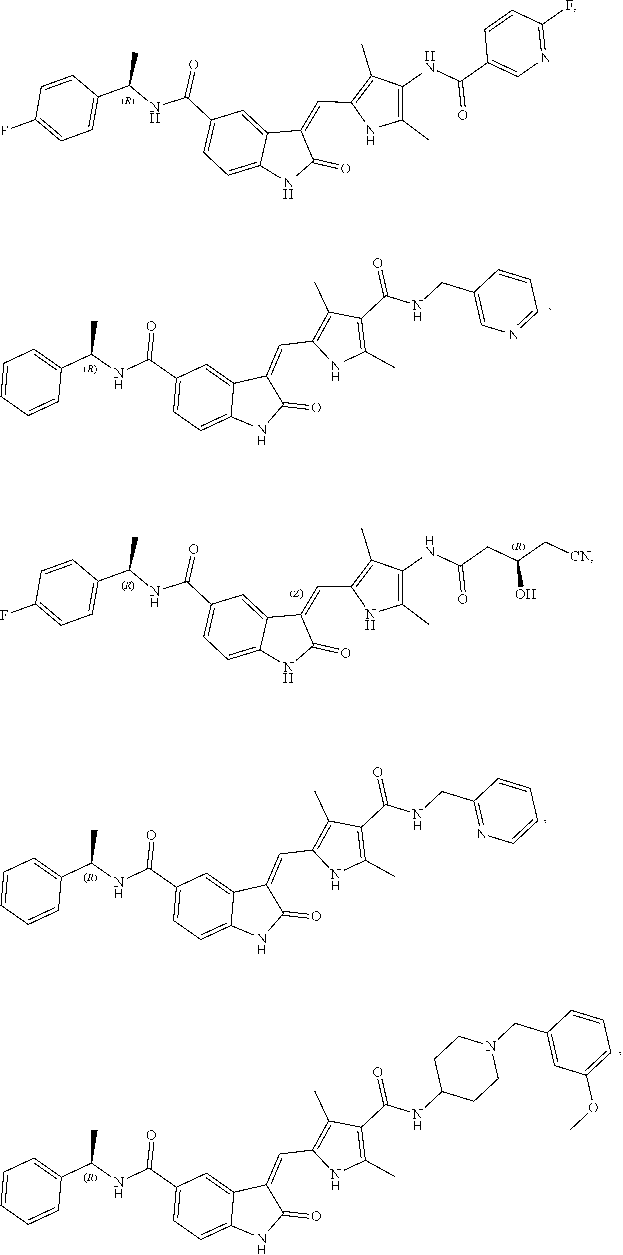

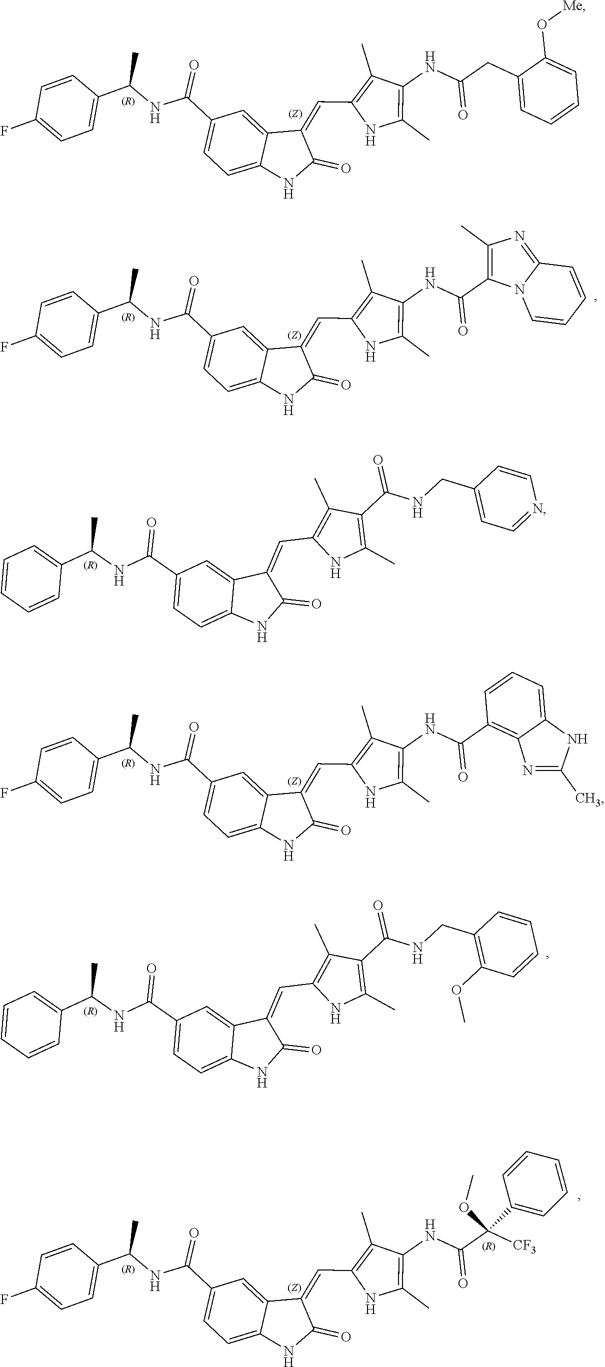

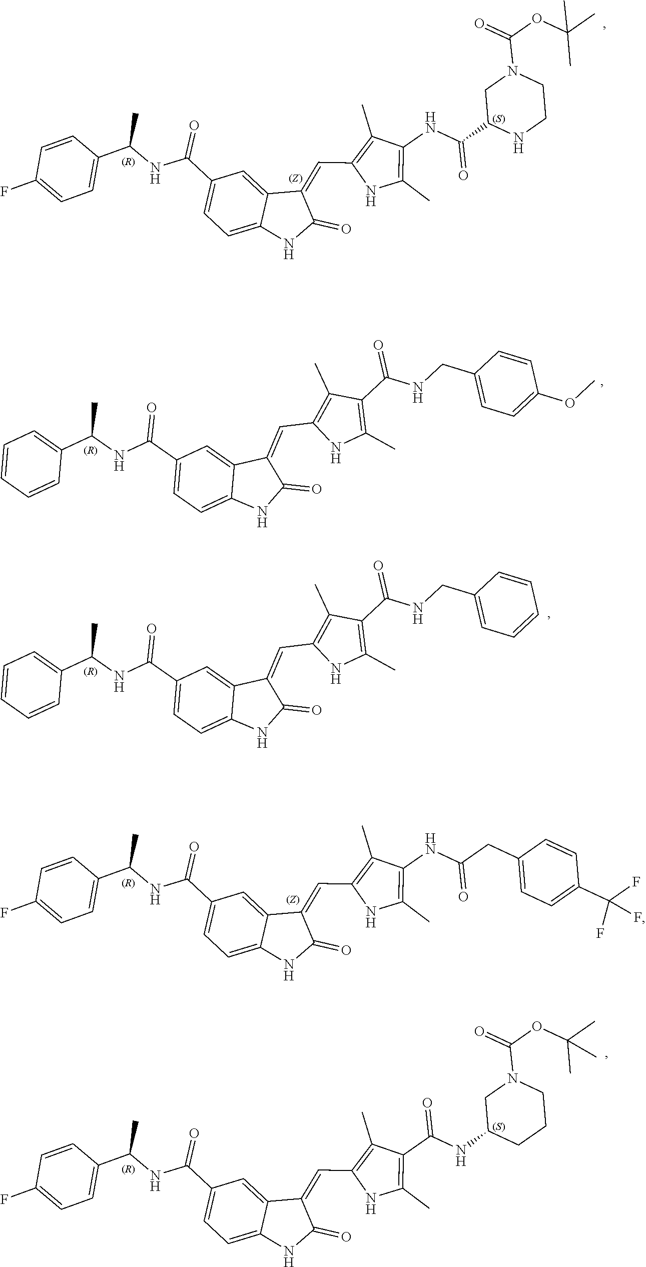

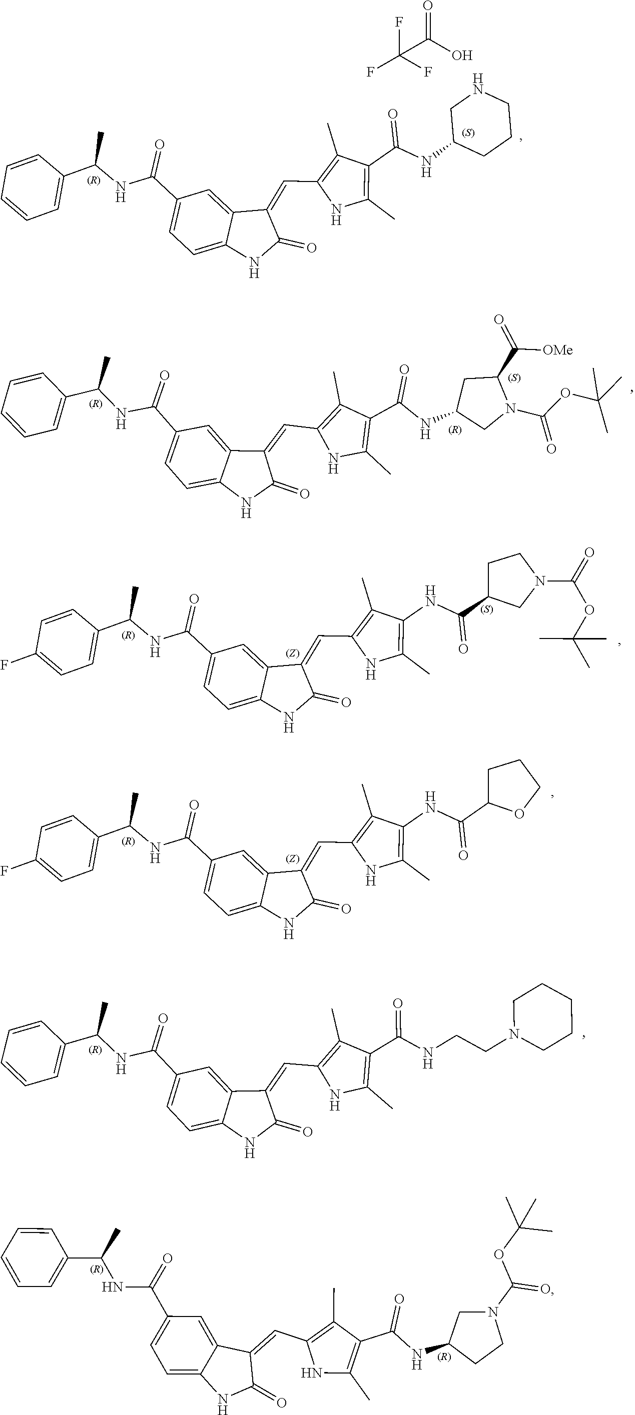

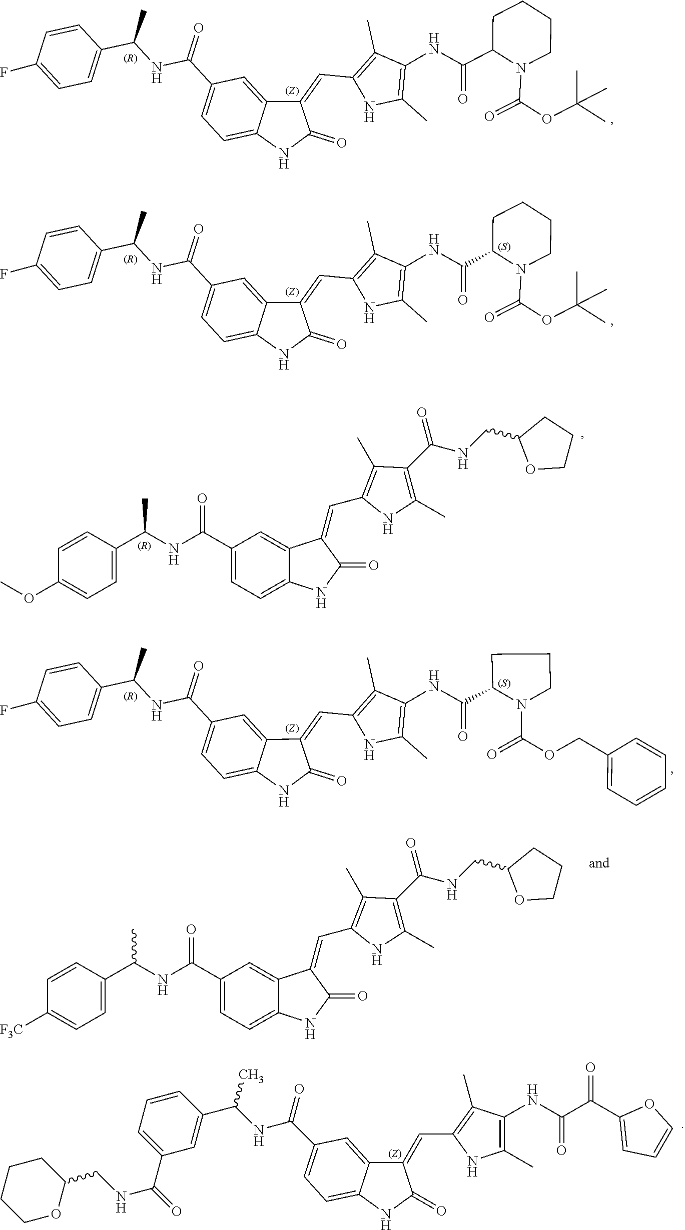

The above compounds can be synthesized in accordance with methods known in the art and exemplified herein. See, e.g., Example 1.

The compound can be a pharmaceutically acceptable salt. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable. Similarly, “pharmaceutically acceptable salt” refers to those salts with counter ions, which may be used in pharmaceuticals. Such salts may include, without limitation, (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethane sulfonic acid, p-toluene sulfonic acid, salicylic acid, tartaric acid, citric acid, succinic acid or malonic acid and the like; or (2) salts formed when an acidic proton present in the parent compound either is replaced by a metal ion, e.g., an alkali metal ion, an alkaline earth ion, or an aluminum ion, or coordinates with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Acceptable salts can be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds can be formed from bases that form non-toxic salts. Illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemi-salts of acids and bases, such as hemi-sulphate and hemi-calcium salts, also can be formed.

One of ordinary skill in the art will further appreciate that the above compounds can be “deuterated,” meaning one or more hydrogen atoms can be replaced with deuterium. As deuterium and hydrogen have nearly the same physical properties, deuterium substitution is the smallest structural change that can be made.

The compounds, in some embodiments, can contain one or more asymmetric centers and thus give rise to enantiomers, diastereomers, and other stereoisomeric forms that are defined, in terms of absolute stereochemistry, as (R) or (S). Unless stated otherwise, it is intended that all stereoisomeric forms of the compounds are contemplated. When the conjugates contain alkene double bonds, and unless specified otherwise, it is intended that both E and Z geometric isomers (e.g., cis or trans) are included. Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Further provided is a pharmaceutical composition comprising an above-described compound and a pharmaceutically acceptable carrier or excipient. The term “composition” generally refers to any product comprising more than one ingredient, including the compound. It is to be understood that the compositions can be prepared from isolated compounds or from salts, solutions, hydrates, solvates, and other forms of the compounds. It is appreciated that certain functional groups, such as the hydroxy, amino, and like groups can form complexes with water and/or various solvents, in the various physical forms of the compound. It is also to be understood that the compositions can be prepared from various amorphous, non-amorphous, partially crystalline, crystalline, and/or other morphological forms of the compounds, and the compositions can be prepared from various hydrates and/or solvates of the compounds.

Accordingly, such pharmaceutical compositions can include each of, or any combination of, or individual forms of, the various morphological forms and/or solvate or hydrate forms of the compounds.

Any pharmaceutically acceptable carriers and excipients as known in the art can be used. A pharmaceutically acceptable carrier can include a solvent, dispersion medium, a coating, an antibacterial and/or antifungal agent(s), an isotonic and/or absorption delaying agent(s), and the like, and combinations thereof, that are physiologically compatible. The carrier can be suitable for parenteral administration, e.g., a sterile aqueous solution or dispersion or a sterile powder for the extemporaneous preparation of a sterile injectable solution or dispersion.

Examples of various ingredients include, but are not limited to, a color additive, a preservative, and a stabilizer. More specific examples include crystal cellulose, calcium carmellose, sodium carmellose, hydropropylcellulose, hydroxypropylmethylcellulose, ethylcellulose, and magnesium stearate. Such compositions can be manufactured in accordance with methods in the art and described, for example, in Remington, The Science and Practice of Pharmacy, 22nd edition. Supplementary active compounds can also be incorporated into the compositions.

Oral dosage units can be tablets or capsules, for example. Other compositions for oral administration include elixirs, syrups, and the like.

Solutions of the active composition can be aqueous, optionally mixed with a nontoxic surfactant and/or can contain carriers or excipients, such as salts, carbohydrates and buffering agents (preferably at a pH of from 3 to 9), but, for some applications, they can be more suitably formulated as a sterile non-aqueous solution or as a dried form to be used in conjunction with a suitable vehicle, such as sterile, pyrogen-free water or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

Excipients can include suspending agents, for example, sodium carboxymethylcellulose, methylcellulose, hydroxypropylmethylcellulose, sodium alginate, polyvinylpyrrolidone, gum tragacanth and gum acacia; dispersing or wetting agents, which can be a naturally occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxcycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions can also contain one or more preservatives, for example ascorbic acid, ethyl, n-propyl, or p-hydroxybenzoate; or one or more coloring agents.

Dispersible powders and granules suitable for preparation of an aqueous suspension by the addition of water can provide the active ingredient in admixture with a dispersing or wetting agent, suspending agent and one or more preservatives. Additional excipients, for example, coloring agents, can also be present.

Suitable emulsifying agents can be naturally occurring gums, for example, gum acacia or gum tragacanth; naturally occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate. Isotonic agents, for example, sugars, polyalcohols, such as mannitol or sorbitol, or sodium chloride can be included in the composition. Prolonged absorption of injectable compositions can be brought about by including in the composition an agent which delays absorption, such as monostearate salts and gelatin.

Liquid formulations can include suspensions and solutions. Such formulations can comprise a carrier, for example, water, ethanol, polyethylene glycol, propylene glycol, methylcellulose, or a suitable oil, and one or more emulsifying agents and/or suspending agents. Liquid formulations can also be prepared by the reconstitution of a solid.

Still further provided is a method of treating a subject in need of inhibition of G protein-coupled receptor kinase 5 (GRK5). The method comprises administering to the subject in need thereof an effective amount of an above-described compound or a pharmaceutical composition comprising an above-described compound and a pharmaceutically acceptable carrier or excipient. In an embodiment, the subject may have heart disease (e.g., hypertrophic cardiomyopathy or heart failure). In another embodiment, the subject may have cancer.

Also contemplated herein is one or more compounds described herein for use as a medicament for treating a patient in need of inhibition of G protein-coupled receptor kinase 5 (GRK5). The subject may have heart disease (e.g., hypertrophic cardiomyopathy or heart failure) and/or cancer.

The compound can be formulated as a pharmaceutical composition and administered to a subject, such as a mammal, e.g., a human, in a variety of forms adapted to the chosen route of administration as discussed above. For example, the composition can be administered as an oral dosage unit, an injectable composition (i.e., for subcutaneous or intravenous injection), or an infusion. See, e.g., Remington, supra.

An effective amount of the compound, or the pharmaceutical composition comprising the compound, can be determined in accordance with methods known in the art (e.g., animal models, human data, and human data for compounds that are used in a similar manner). The amount can be determined by taking into consideration various factors, such as the potency of the conjugate, body weight, mode of administration, the type and location of fracture, and its causation. The effective amount can range from about 0.1 μg/kg/day, such as 0.5 μg/kg/day, 0.7 μg/kg/day, or 0.01 mg/kg/day up to about 1,000 mg/kg/day. Intravenous doses can be several orders of magnitude lower. The compound/composition can be administered more than once, such as daily (1-3 or more times per day), weekly (including 1-3 or more times on a given day), bi-weekly (including 1-3 or more times on a given day), monthly (including 1-3 or more times on a given day), or bimonthly (including 1-3 or more times on a given day).

The terms “substituted,” “substituent,” and “functional group” refer to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

The term “alkyl” as used herein refers to substituted or unsubstituted straight chain and branched mono- or divalent alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and ante-isoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched mono- or divalent alkenyl groups and cycloalkenyl groups having at least one double bond and having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkenyl groups include those with from 1 to 8 carbon atoms such as —CH═CH—, —CH═CHCH₃, and —CH₂CH═CHCH₂— groups, wherein the double bonds can have an E- or Z-configuration. And when there are multiple bonds, each double bond can, independently, have an E- or a Z-configuration. Examples of branched alkenyl groups include, but are not limited to, —CH═C(CH₃)— and CH₂C═CH(CH₃) groups. Representative substituted alkenyl groups can be substituted one or more times with any of the groups listed herein, for example, amino, hydroxy, cyano, carboxy, nitro, thio, alkoxy, and halogen groups.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups can have any number of carbon atoms, e.g., 3 to 8 carbon atoms (C₃-C₈), 3 to 6 carbon atoms (C₃-C₆), and 4 to 8 carbon atoms (C₄-C₈). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “cycloalkylalkyl” as used herein refers to substituted or unsubstituted alkyl groups as defined herein in which a hydrogen or carbon bond of an alkyl group as defined herein is replaced with a bond to a cycloalkyl group as defined herein. Representative cycloalkylalkyl groups include, but are not limited to, cyclopentylalkyl.

The term “alkylcycloalkyl” as used herein refers to substituted or unsubstituted cycloalkyl groups as defined herein in which a hydrogen of a cycloalkyl group as defined herein is replaced with a bond to an alkyl group as defined herein. Representative alkylcycloalkyl groups include, but are not limited to, alkylcyclopropyl.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclylcarbonyl group is a prolyl group, wherein the prolyl group can be a D- or an L-prolyl group.

The term “aryl” as used herein refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. “Aryl” and the phrase “aryl group” includes fused ring species including those that include fused aromatic and non-aromatic groups. Accordingly, “aryl” and the phrase “aryl group” include groups of the formula:

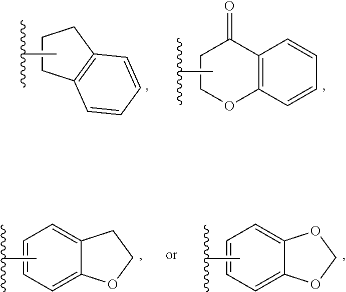

each of which can be substituted or unsubstituted, such as hydroxy substituted.

Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” refers to substituted or unsubstituted aromatic and non-aromatic ring compounds containing 3 or more ring members, of which one or more (e.g., 1, 2 or 3) is a heteroatom such as, but not limited to, N, O, and S. Thus, a heterocyclyl can be a cycloheteroalkyl or a heteroaryl or, if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆), 3 to 5 carbon atoms (C₃-C₅) or 6 to 8 carbon atoms (C₆-C₈). A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds, such as in the group 3,6-dihydro-2H-pyran and 3,4-dihydro-2H-pyran, having the formula:

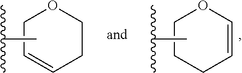

respectively, each of which can be substituted.

A heteroaryl ring is an embodiment of a heterocyclyl group. The phrase “heterocyclyl group” includes fused ring species including those that include fused aromatic and non-aromatic groups. Representative heterocyclyl groups include, but are not limited to tetrahydro-2H-thiopyran-1,1-dioxide, having the formula:

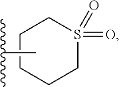

which can be substituted, 4a,5,6,7-tetrahydro-4H-pyrrolo[1,2-d][1,3,4]oxadiazinyl, having the formula:

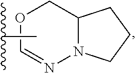

which can be substituted, pyrrolidinyl, pyrrolidinone (e.g., pyrrolidin-2-one), azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, imidazo[1,2-a]pyridinyl, having the formula:

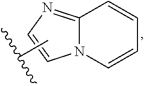

which can be substituted, triazyolyl, tetrazolyl, benzoxazolinyl, thiazolyl, benzthiazolinyl, and benzimidazolinyl groups. Examples of indolinonyl groups include groups having the general formula:

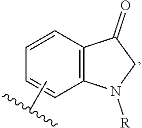

wherein R is as defined herein.

Examples of isoindolinonyl groups include groups having the general formula:

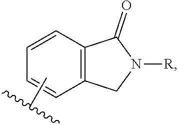

wherein R is as defined herein.

Examples of benzoxazolinyl groups include groups having the general formula:

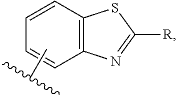

wherein R is as defined herein.

Examples of benzthiazolinyl groups include groups having the general formula:

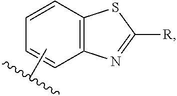

wherein R is as defined herein.

In some embodiments, the group R in benzoxazolinyl and benzthiazolinyl groups is an N(R)₂ group. In some embodiments, each R is hydrogen or alkyl, wherein the alkyl group is substituted or unsubstituted. In some embodiments, the alkyl group is substituted with a heterocyclyl group (e.g., with a pyrrolidinyl group).

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heterocyclylalkoxy” refers to alkyl groups as defined herein in which a hydrogen or carbon bond of an alkyl group as defined herein is replaced with a bond to a heterocyclyl group as defined herein and the alkyl group is attached to an oxygen. Representative heterocyclylalkoxy groups include, but are not limited to, —O—(CH₂)_{q}heterocyclyl, wherein q is an integer from 1 to 5. In some embodiments, heterocyclylalkoxy groups include —O—(CH₂)_{q}morpholinyl such as —O—CH₂CH₂-morpholine.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The terms “amine,” “amine group,” “amino,” and “amino group” refer to a substituent of the form —NH₂, —NHR, —NR₂, or —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group.

An “alkylamino” group includes a monoalkylamino, dialkylamino, and trialkylamino group. An example of a “alkylamino” is —NH-alkyl and —N(alkyl)₂.

An example of a “cycloalkylamino” group is —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The term “amido” refers to a group of the formula —C(O)NR₂, wherein R is defined herein.

The terms “halo,” “halogen,” and “halide” group, by themselves or as part of another substituent, mean, unless otherwise stated, a fluorine, chlorine, bromine, or iodine atom.

The term “haloalkyl” group includes mono-halo alkyl groups, poly-halo alkyl groups, wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference for its teachings regarding same.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery, 6th ed. (Donald J. Abraham ed., 2001, Wiley), and Design and Application of Prodrugs (H. Bundgaard ed., 1985, Harwood Academic Publishers GmbH).

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The disclosure can be better understood by reference to the following examples which are offered by way of illustration. The disclosure is not limited to the examples given herein.

### Example 1

### Preparation of Compound 1

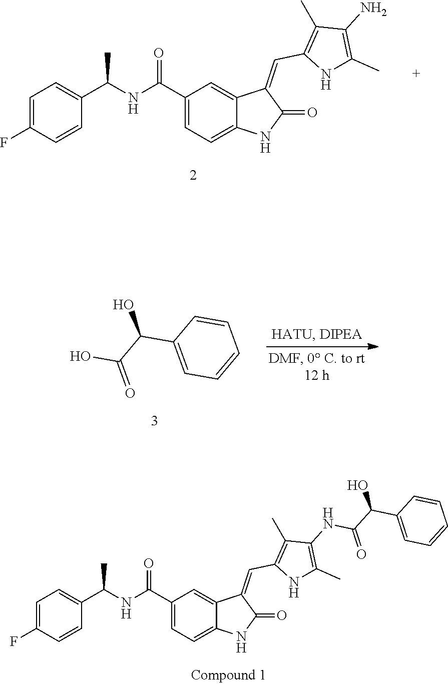 [SMILES: Cc1[nH]c(/C=C2\C(=O)Nc3ccc(C(=O)N[C@H](C)c4ccc(F)cc4)cc32)c(C)c1N ; O=C(O)[C@@H](O)c1ccccc1 ; Cc1[nH]c(/C=C2\C(=O)Nc3ccc(C(=O)N[C@H](C)c4ccc(F)cc4)cc32)c(C)c1NC(=O)[C@@H](O)c1ccccc1]

The synthesis of compound 2 was carried out using literature report (“Generation of Highly Selective, Potent, and Covalent G Protein-Coupled Receptor Kinase 5 Inhibitors,” Rowlands, R. A.; Chen, Q.; Bouley, R. A.; Avramova, L. V.; Tesmer, J. J. G.; White, A. D. J. Med. Chem. 2021, 64, 566-585). Compound 3 was obtained from commercial source.

To a solution of 2 (60 mg, 0.14 mmol, 1 equiv.) in dry DMF (2.5 mL), (S)-mandelic acid 3 (26 mg, 0.17 mmol, 1.1 equiv.), HATU (63 mg, 0.17 mmol, 1.2 equiv.) and DIPEA (37 mg, 0.29 mmol, 2 equiv.) were added at 0° C. The reaction was stirred at room temperature for 12 h. After completion, the reaction mixture was extracted with ethyl acetate and brine, purified using flash column chromatography (DCM:MeOH) (94:6), furnishing compound 1 (24 mg) as a red colored solid. Yield: 49%; ¹H NMR (400 MHz, DMSO): δ 11.03 (s, 1H), 9.28 (s, 1H), 8.58 (d, J=8.0 Hz, 1H), 8.17 (d, J=1.2 Hz, 1H), 7.65 (dd, J=8.1, 1.6 Hz, 1H), 7.60 (s, 1H), 7.57-7.47 (m, 2H), 7.42 (dd, J=8.6, 5.6 Hz, 2H), 7.36 (dd, J=8.0, 6.7 Hz, 2H), 7.29 (d, J=7.3 Hz, 1H), 7.13 (t, J=8.9 Hz, 2H), 6.90 (d, J=8.1 Hz, 1H), 6.28 (d, J=4.7 Hz, 1H), 5.24-5.11 (m, 1H), 5.09 (d, J=4.7 Hz, 1H), 2.09 (s, 6H), 1.47 (d, J=7.1 Hz, 3H); Chemical formula of Compound 1: C₃₂H₂₉FN₄O₄; ESI-MASS Calc: 522.22 found [M+H]+.

### Preparation of Compounds 2 and 3

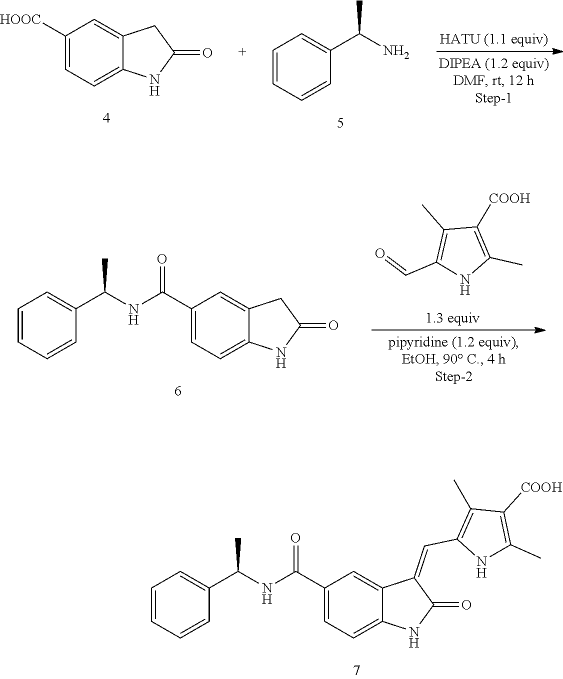

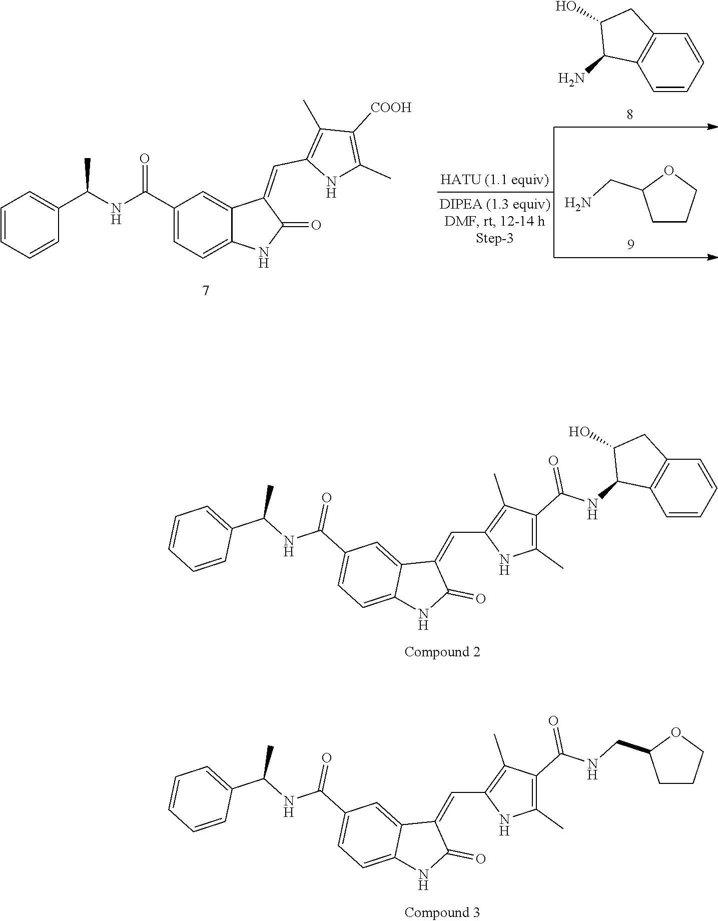

### Synthesis of Compound 6

To a stirred solution of compound 5-carboxyindoline 4 (438 mg, 2.47 mmol, 1.2 equiv) in dry DMF (4 mL) was added with (R)-1-phenylethan-1-amine 5 (0.26 mL, 2.06 mmol, 1 equiv), HATU (904 mg, 2.47 mmol, 1.2 equiv) and DIPEA (0.46 mL, 2.7 mmol, 1.3 equiv) under Ar atmosphere at 0° C. the mixture was stirred for 12 h at 23° C. After completion of the reaction (monitored by TLC), the mixture was quenched with sat. Na₂CO₃ (10 mL) and the mixture was extracted with EtOAc (3×50 mL). The combined organic layer was washed with brine (2×50 mL) and then dried over NaSO₄ and concentrated under reduced presser. The crude mixture was purified by column chromatography (0-15% MeOH in DCM) to give compound 6 as a pale pink color solid in 89% yields.

### Synthesis of Compound 7

To a stirred solution of compound 6 (100 mg, 0.41 mmol, 1 equiv) in absolute ethanol (2 mL), 5-formyl-2,4-dimethyl-1H-pyrrole-3-carboxylic acid (151 mg, 0.48 mmol, 1.1 equiv) was added. To this solution piperidine (0.04 mL, 0.497 mmol, 1.2 equiv) was added and the resulting mixture was heated to reflux (90° C.) for 4 h. After completion, the reaction was cooled to room temperature and the product was filtered off, the collected orange solid compound 7 was washed with cold ether and dried under vacuum, yield 78%. This compound was used for the next reaction without further purification.

### Synthesis of Compound 2

To a stirred solution of compound 7 (30 mg, 0.06 mmol) in dry DMF (1 mL) was added with (1R,2R)-1-amino-2,3-dihydro-1H-inden-2-ol 8 (8.4 mg, 0.06 mmol), HATU (22.7 mg, 0.06 mmol) and DIPEA (12 μL, 0.07 mmol) under Argon atmosphere at 0° C. and the mixture was stirred for 12-14 h at 23° C. After completion of the reaction (monitored by TLC), the solvent was evaporated under reduced pressure and the crude mixture was diluted with water and extracted with DCM (3×10 mL). The combined organic layer was dried over NaSO₄ and concentrated under reduced presser. The resulted crude mixture was purified by column chromatography (0-5% MeOH in DCM) to give Compound 2 as an orange solid in 61% (18 mg) yields. ¹H NMR (400 MHz, DMSO-d6) δ 11.14 (s, 1H), 8.62 (d, J=7.9 Hz, 1H), 8.26 (s, 1H), 8.02 (d, J=8.6 Hz, 1H), 7.71 (d, J=4.8 Hz, 2H), 7.42 (d, J=7.5 Hz, 2H), 7.34 (t, J=7.6 Hz, 2H), 7.27-7.17 (m, 5H), 6.95 (d, J=8.1 Hz, 1H), 5.35 (d, J=5.8 Hz, 1H), 5.28 (t, J=7.7 Hz, 1H), 5.23-5.15 (m, 1H), 4.42-4.34 (m, 1H), 3.17 (dd, J=15.5, 7.3 Hz, 1H), 2.75 (dd, J=15.3, 7.7 Hz, 1H), 2.56-2.42 (m, 6H), 1.52 (d, J=7.0 Hz, 3H). Chemical Formula of Compound 2: C₃₄H₃₂N₄O₄; ESI-MASS Calc: 560.65 found [M+H]⁺.

### Synthesis of Compound 3

To a stirred solution of compound 7 (30 mg, 0.06 mmol) in dry DMF (1 mL) was added with (tetrahydrofuran-2-yl)methanamine 9 (6.3 mg, 0.06 mmol), HATU (22.7 mg, 0.06 mmol) and DIPEA (12 μL, 0.07 mmol) under Argon atmosphere at 0° C. and the mixture was stirred for 12-14 h at 23° C. After completion of the reaction (monitored by TLC), the solvent was evaporated under reduced pressure and the crude mixture was diluted with water and extracted with DCM (3×10 mL). The combined organic layer was dried over NaSO₄ and concentrated under reduced presser. The resulted crude mixture was purified by column chromatography (0-3% MeOH in DCM) to give compound 3 as an orange solid in 64% (19 mg) yields. ¹H NMR (400 MHz, DMSO-d6) δ 11.11 (s, 1H), 8.58 (d, J=8.0 Hz, 1H), 8.22 (s, 1H), 7.73-7.63 (m, 3H), 7.39 (d, J=7.3 Hz, 2H), 7.31 (t, J=7.6 Hz, 2H), 7.20 (t, J=7.3 Hz, 1H), 6.91 (d, J=8.1 Hz, 1H), 5.21-5.13 (m, 1H), 3.96-3.91 (m, 1H), 3.79-3.74 (m, 1H), 3.62 (d, J=6.8 Hz, 1H), 3.26 (d, J=5.0 Hz, 2H), 2.41 (d, J=6.6 Hz, 6H), 1.87 (ddd, J=23.4, 13.6, 7.2 Hz, 3H), 1.63-1.56 (m, 1H), 1.48 (d, J=7.1 Hz, 3H). Chemical Formula of Compound 3: C₃₀H₃₂N₄O₄ ESI-MASS Calc: 512.61 found [M+H]⁺.

Other compounds described herein can be synthesized in a manner similar to the way as the compounds described in this Example.

### Example 2

### GRK2 and GRK5 Inhibition Assays

For all new synthetic compounds, IC₅₀ values for human GRK5 and bovine GRK2 were determined using a radiometric assay, described by us in the literature (Rowlands et al. (2021), supra). Published procedure: GRK (50 nM) was incubated for 3-5 min with 500 nM porcine brain tubulin (PurSolutions) and 0.01-50 μM new synthetic inhibitor in 20 mM HEPES (pH 7.0), 2 mM MgCl₂, 0.025% dodecylmaltoside (DDM), and 1% DMSO prior to initiation with the addition of 5 μM ATP supplemented with radioactive [γ-³²P]-ATP (PerkinElmer Life Sciences). Reactions were quenched at 8 min by addition of 5 μL of 4×SDS gel loading dye to the 10 μL reactions. Samples (12 μL) were separated on a 4-15% Criterion TGX precast gel (Bio-Rad). For potent inhibitors with low nanomolar IC₅₀, the inhibitor concentration was adjusted to approximately 0-50× [IC₅₀], which was estimated from the first run to get more accurate measurements. Gels were dried, exposed to a storage phosphor screen overnight, and scanned using a Personal Molecular Imager (Bio-Rad). Bands corresponding to phosphorylated tubulin were quantified using ImageQuant, plotted as a function of log[inhibitor], and fit to the four-parameter log(inhibitor) vs response model in GraphPad Prism 7.03 to determine the IC₅₀, mean, and standard deviation values. Outliers were eliminated automatically at a 1% Q value. Experiments were performed at least three times.

PKA inhibition assays were performed with the ADP-Glo system (Promega Corporation) according to the manufacturer's instructions. PKA (500 nM) was incubated with 1 μg of CREBtide (KRREILSRRPSYR) (Genscript Corporation) substrate, 50 μM ATP, and inhibitor for 30 min in 20 mM HEPES (pH 7.0), 2 mM MgCl₂, 0.025% dodecylmaltoside (DDM), and 4% DMSO. The concentration range of each inhibitor varies depending on its solubility at 4% DMSO with the highest concentration from 100 to 500 μM. After the initial reaction, ADP-Glo reagent was added to the reaction and allowed to incubate for an additional 40 min. Last, the kinase detection reagent was added and allowed to incubate for 30 min, and luminescence was measured with a FlexStation 3 Multi-mode Microplate Reader (Molecular Devices). All data was analyzed in the same way as in the GRK inhibition assay. Experiments were performed three times in duplicate.

Standard control compounds are run during each assay to assess consistency across time, experimenters, and subtle changes in assay conditions that are sometimes required to keep compounds soluble and dispersed (e.g., through addition of DDM or 3% DMSO). Paroxetine was used as a control for GRK2 (“Paroxetine is a direct inhibitor of g protein-coupled receptor kinase 2 and increases myocardial contractility,” Thal, D. M.; Homan, K. T.; Chen, J.; Wu, E. K.; Hinkle, P. M.; Huang, Z. M.; Chuprun, J. K.; Song, J.; Gao, E.; Cheung, J. Y.; Sklar, L. A.; Koch, W. J.; Tesmer, J. J. G. ACS Chem. Biol. 2012, 7, 1830-1839) and PKA and CCG215022 for GRK5 (“Crystal Structure of G Protein-coupled Receptor Kinase 5 in Complex with a Rationally Designed Inhibitor,” Homan, K. T.; Waldschmidt, H. V.; Glukhova, A.; Cannavo, A.; Song, J.; Cheung, J. Y.; Koch, W. J.; Larsen, S. D.; Tesmer, J. J. J. Biol. Chem. 2015, 290, 20649-20659).

List of Compounds, Structures, in vitro GRK2 and GRK5 inhibition data range

| | GRK5 | GRK2 |
|---|---|---|
| Structure | (IC₅₀) | (IC₅₀) |
| 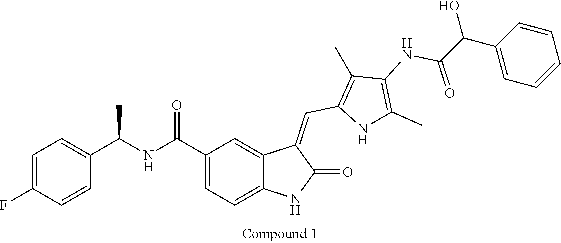 [SMILES: Cc1[nH]c(/C=C2\C(=O)Nc3ccc(C(=O)N[C@H](C)c4ccc(F)cc4)cc32)c(C)c1NC(=O)C(O)c1ccccc1] | <100 nM (++++) | >10 μM (+) |
| 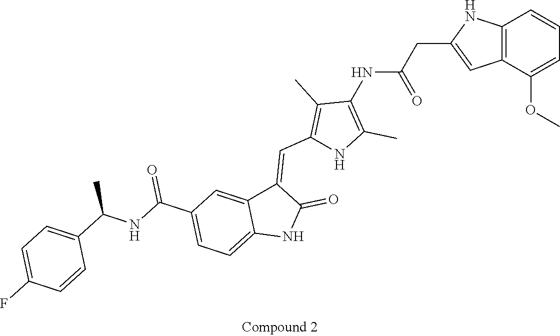 [SMILES: COc1cccc2[nH]c(CC(=O)Nc3c(C)[nH]c(/C=C4\C(=O)Nc5ccc(C(=O)N[C@H](C)c6ccc(F)cc6)cc54)c3C)cc12] | ++ | + |
| 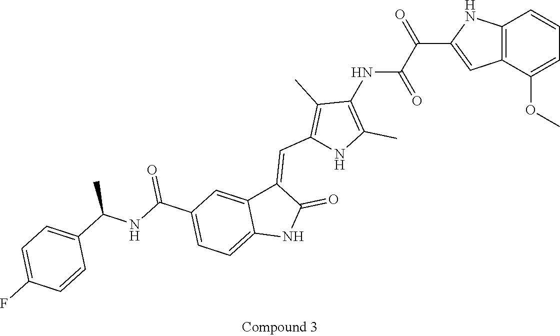 [SMILES: COc1cccc2[nH]c(C(=O)C(=O)Nc3c(C)[nH]c(/C=C4\C(=O)Nc5ccc(C(=O)N[C@H](C)c6ccc(F)cc6)cc54)c3C)cc12] | ++ | + |
| 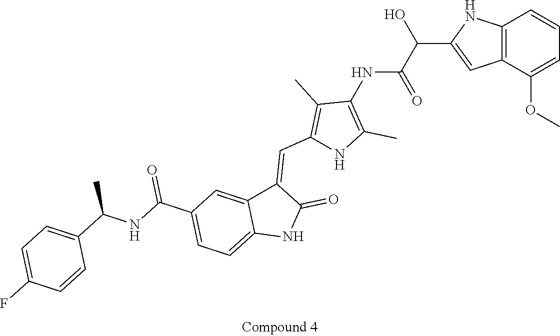 [SMILES: COc1cccc2[nH]c(C(O)C(=O)Nc3c(C)[nH]c(/C=C4\C(=O)Nc5ccc(C(=O)N[C@H](C)c6ccc(F)cc6)cc54)c3C)cc12] | +++ | + |
| 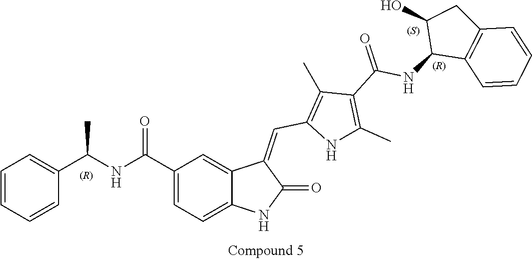 | +++ | + |
| 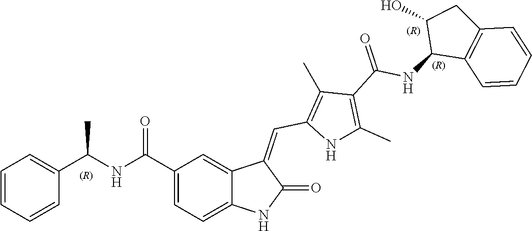 | <999 nM (+++) | >10 μM (+) |
| Compound 6 | | |
| 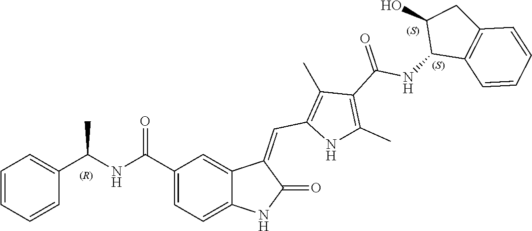 | +++ | + |
| Compound 7 | | |
| 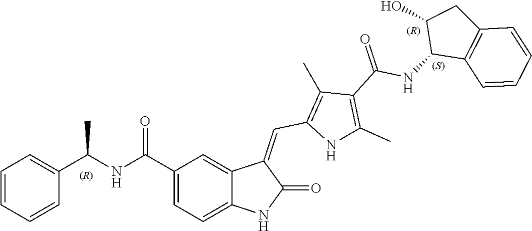 | +++ | + |
| Compound 8 | | |
| 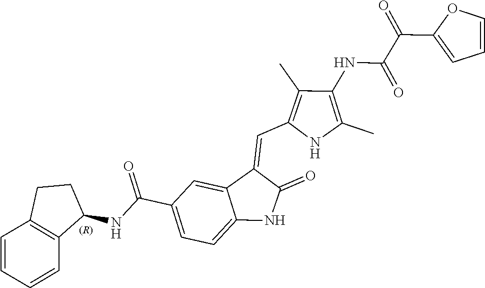 | + | + |
| Compound 9 | | |
| 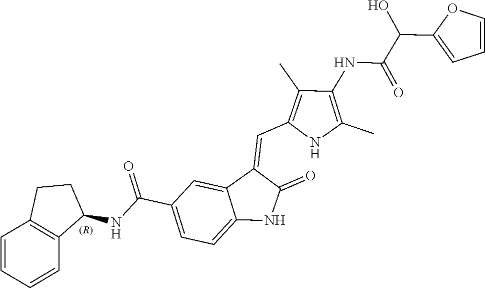 | +++ | + |
| Compound 10 | | |
| 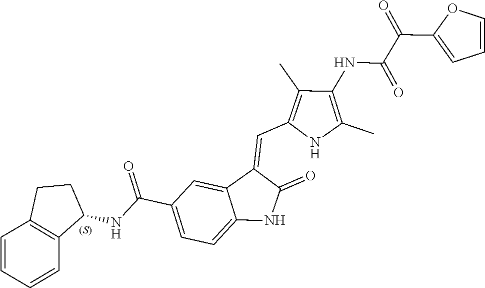 | ++ | + |
| Compound 11 | | |
| 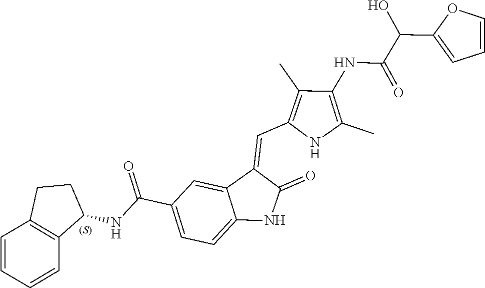 | ++ | + |
| Compound 12 | | |
| 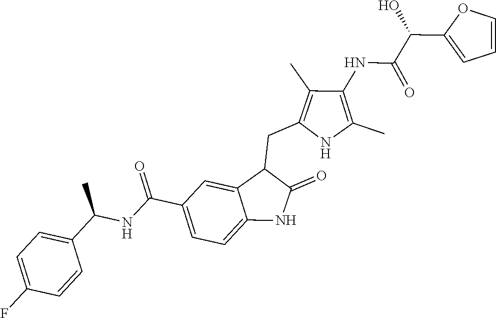 [SMILES: Cc1[nH]c(CC2C(=O)Nc3ccc(C(=O)N[C@H](C)c4ccc(F)cc4)cc32)c(C)c1NC(=O)[C@H](O)c1ccco1] | +++ | + |
| Compound 13 | | |
| 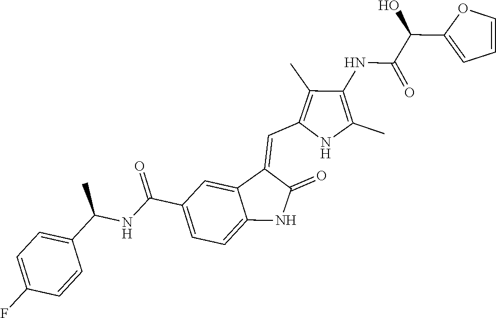 [SMILES: Cc1[nH]c(/C=C2\C(=O)Nc3ccc(C(=O)N[C@H](C)c4ccc(F)cc4)cc32)c(C)c1NC(=O)[C@@H](O)c1ccco1] | ++++ | ++ |
| Compound 14 | | |
| 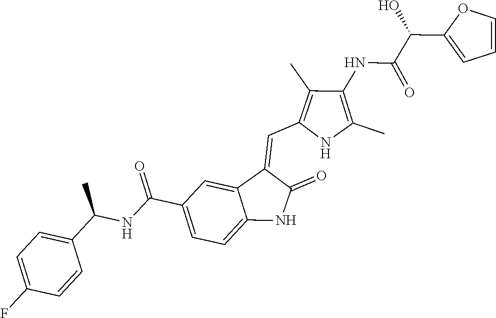 [SMILES: Cc1[nH]c(/C=C2\C(=O)Nc3ccc(C(=O)N[C@H](C)c4ccc(F)cc4)cc32)c(C)c1NC(=O)[C@H](O)c1ccco1] | ++ | ++ |
| Compound 15 | | |
| 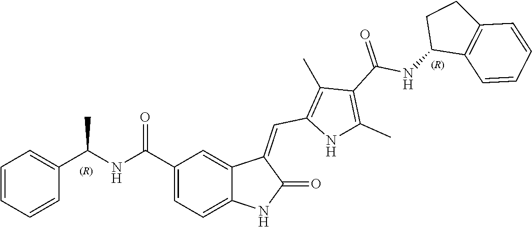 | ++ | + |
| Compound 16 | | |
| 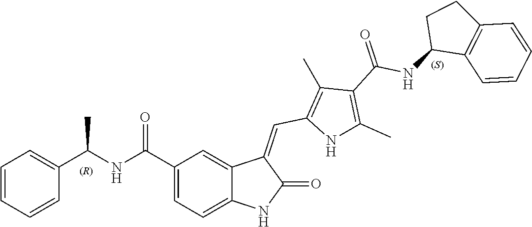 | + | + |
| Compound 17 | | |
| 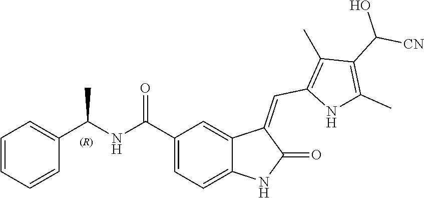 | +++ | + |
| Compound 18 | | |
| 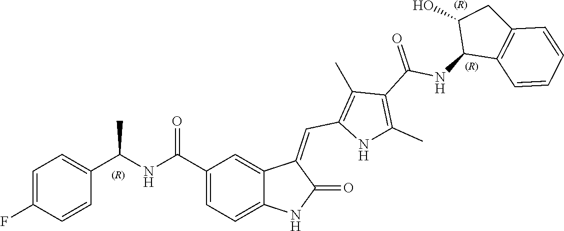 | +++ | + |
| Compound 19 | | |
| 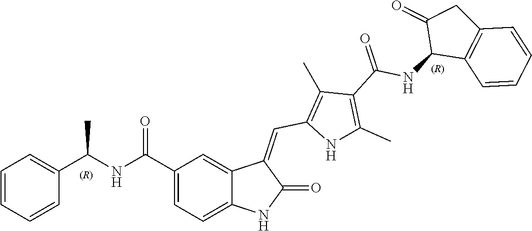 | +++ | + |
| Compound 20 | | |
| 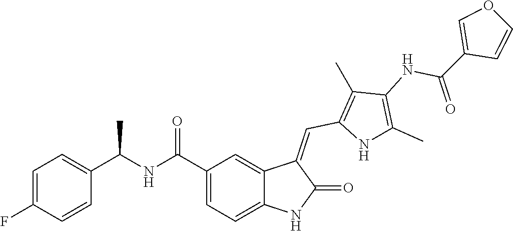 [SMILES: Cc1[nH]c(/C=C2\C(=O)Nc3ccc(C(=O)N[C@H](C)c4ccc(F)cc4)cc32)c(C)c1NC(=O)c1ccoc1] | ++++ | + |
| Compound 21 | | |
| 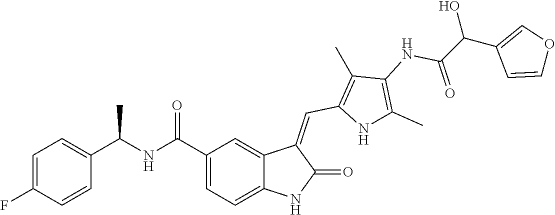 [SMILES: Cc1[nH]c(/C=C2\C(=O)Nc3ccc(C(=O)N[C@H](C)c4ccc(F)cc4)cc32)c(C)c1NC(=O)C(O)c1ccoc1] | ++++ | ++ |
| Compound 22 | | |
| 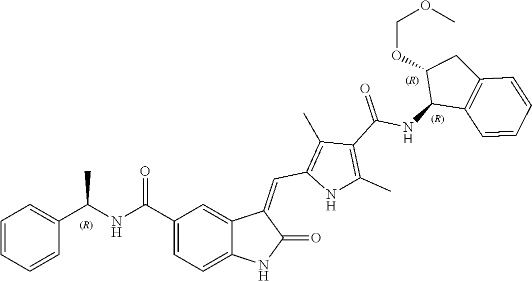 | + | + |
| Compound 23 | | |
| 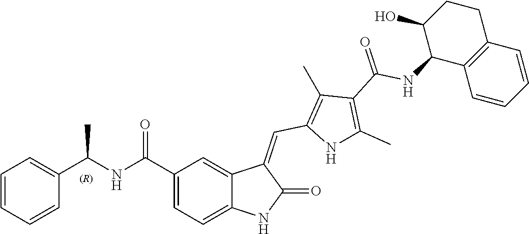 | +++ | + |
| Compound 24 | | |
| 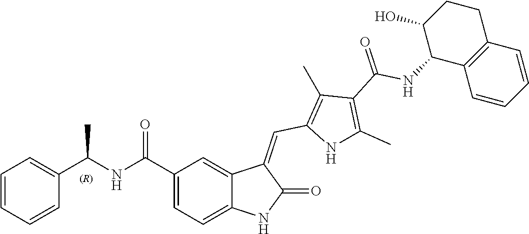 | +++ | + |
| Compound 25 | | |
| 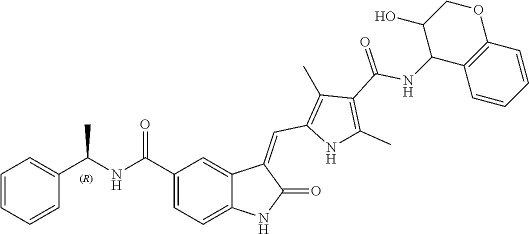 | +++ | + |
| Compound 26 | | |
| 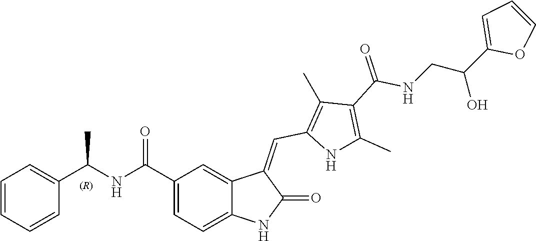 | ++++ | ++ |
| Compound 27 | | |
| 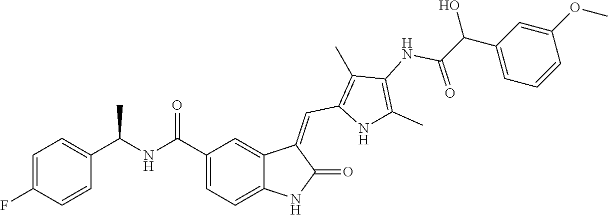 [SMILES: COc1cccc(C(O)C(=O)Nc2c(C)[nH]c(/C=C3\C(=O)Nc4ccc(C(=O)N[C@H](C)c5ccc(F)cc5)cc43)c2C)c1] | +++ | + |
| Compound 28 | | |
| 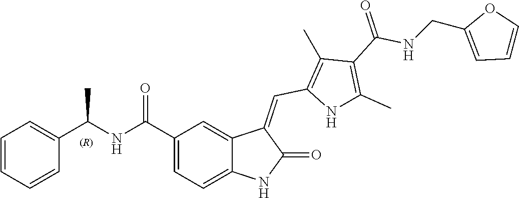 | <100 nM (++++) | <10 μM (++) |
| Compound 29 | | |
| 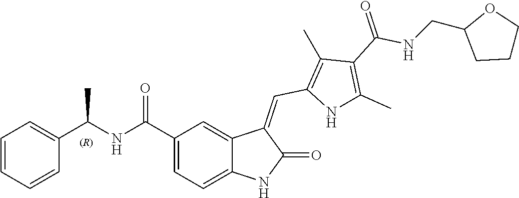 | ++++ | ++ |
| Compound 30 | | |
| 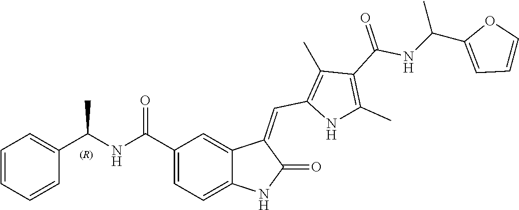 | ++++ | ++ |
| Compound 31 | | |
| 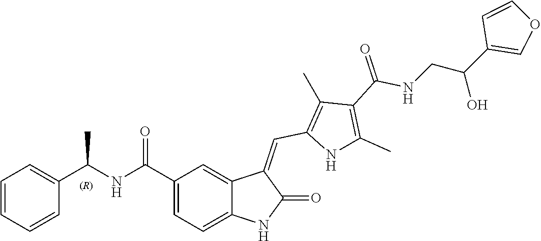 | ++++ | ++ |
| Compound 32 | | |
| 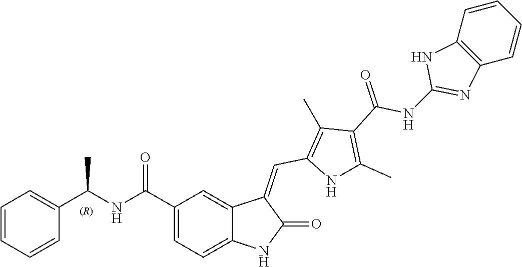 | +++ | ++ |
| Compound 33 | | |
| 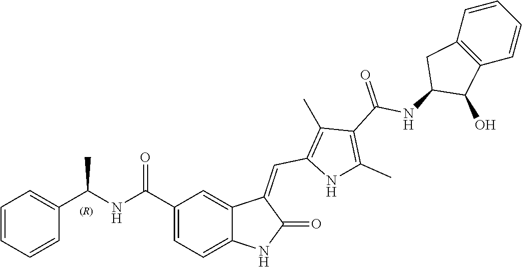 | ++++ | ++ |
| Compound 34 | | |
| 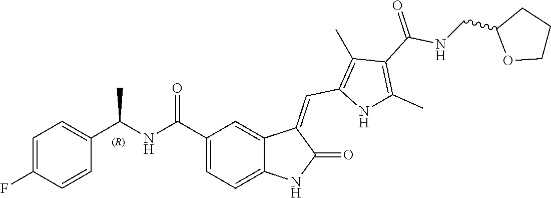 | ++++ | + |
| Compound 35 | | |
| 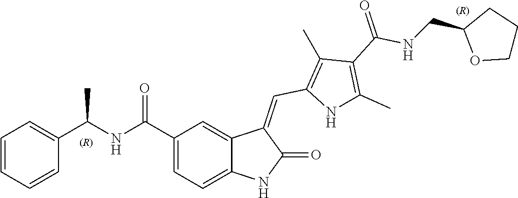 | ++++ | + |
| Compound 36 | | |
| 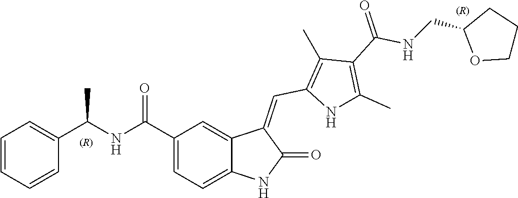 | ++++ | ++ |
| Compound 37 | | |
| 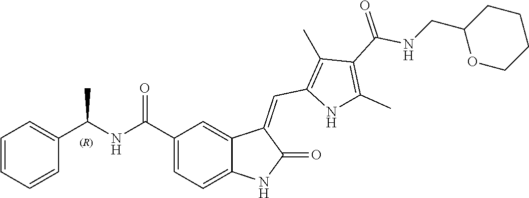 | ++++ | + |
| Compound 38 | | |
| 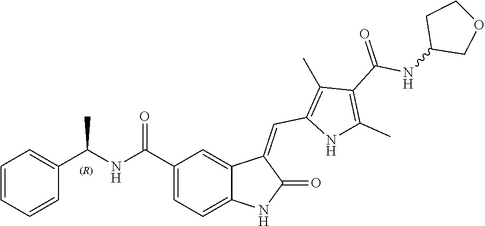 | ++++ | ++ |
| Compound 39 | | |
| 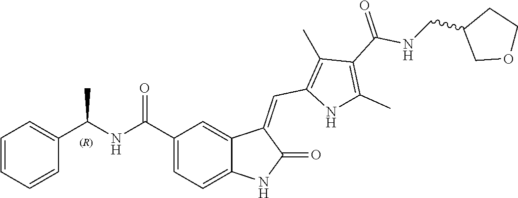 | ++++ | ++ |
| Compound 40 | | |
| 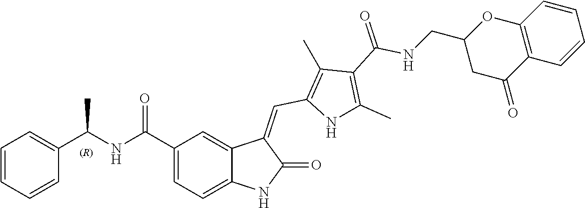 | ++++ | + |
| Compound 41 | | |
| 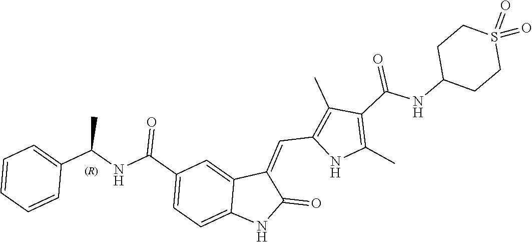 | +++ | + |
| Compound 42 | | |
| 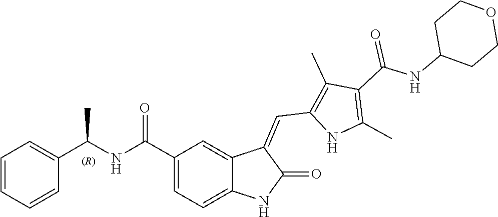 | ++ | + |
| Compound 43 | | |
| 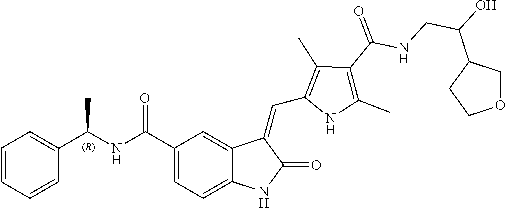 | ++++ | + |
| Compound 44 | | |
| 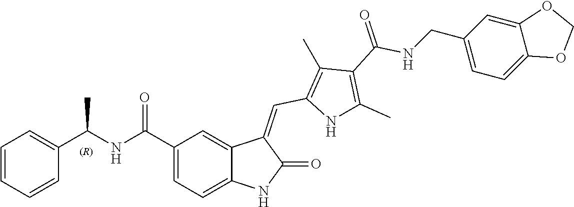 | ND | ND |
| Compound 45 | | |
| 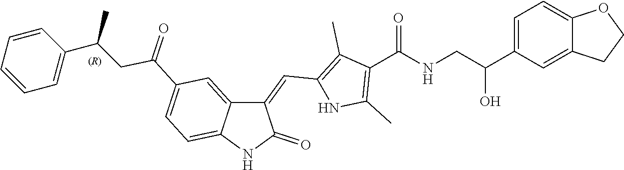 | ND | ND |
| Compound 46 | | |
| 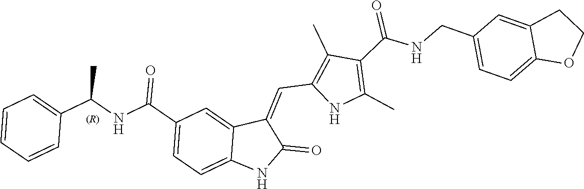 | ND | ND |
| Compound 47 | | |
| 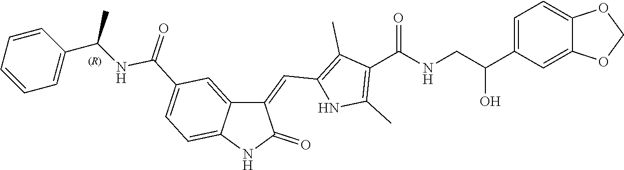 | ND | ND |
| Compound 48 | | |
| 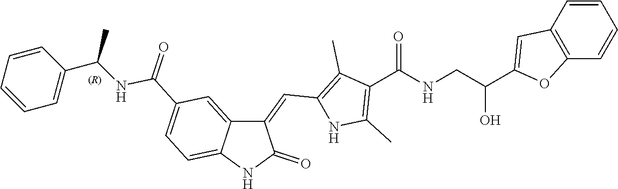 | ND | ND |
| Compound 49 | | |
| 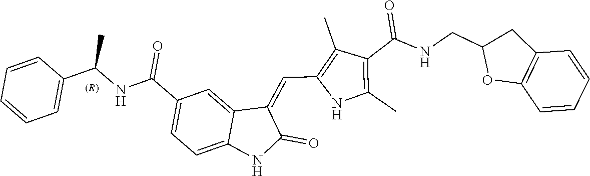 | ND | ND |
| Compound 50 | | |

++++ is IC₅₀ = 1-100 nM;
+++ is IC₅₀ = 101-999 nM;
++ is IC₅₀ = 1-10 μM;
+ is IC₅₀ > 10 μM;
ND = no data

What is claimed is:

1. A compound of the formula: 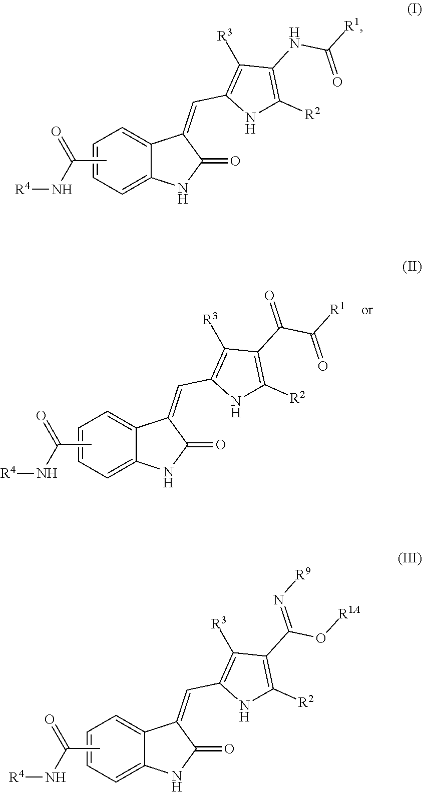
or a pharmaceutically acceptable salt thereof,
wherein:
R¹ is R⁵, -alkyl-R⁵, —C(O)R⁵, 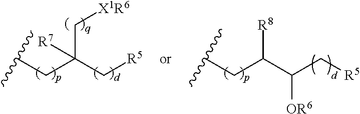
wherein R⁵ is alkyl, hydroxy, alkoxy, amido, cyano, alkenyl, haloalkyl, alkoxyalkyl, aminoalkyl, cycloalkyl, heterocyclyl, aryl or heteroaryl or R⁵ and R⁷ can form a cycloalkyl group;
R^{1A} is H or alkyl or R^{1A} and R⁹ can form a heterocyclyl group;
X¹ is O or NR^{6A} wherein R^{A} is H or alkyl or R^{6A} and R⁵ can form a heterocyclyl group;
p, d, and q are each, independently, an integer from 0 to 5;
R² and R³ are each, independently, alkyl;
R⁶ is H or alkyl;
R⁷ is H, alkyl or aryl;
R⁸ is H or alkyl;
R⁹ is H or alkyl; and
R⁴ is aryl, arylalkyl or heteroarylalkyl.

2. The compound of claim 1, or a pharmaceutically acceptable salt thereof, wherein the compound is a compound of the formula:

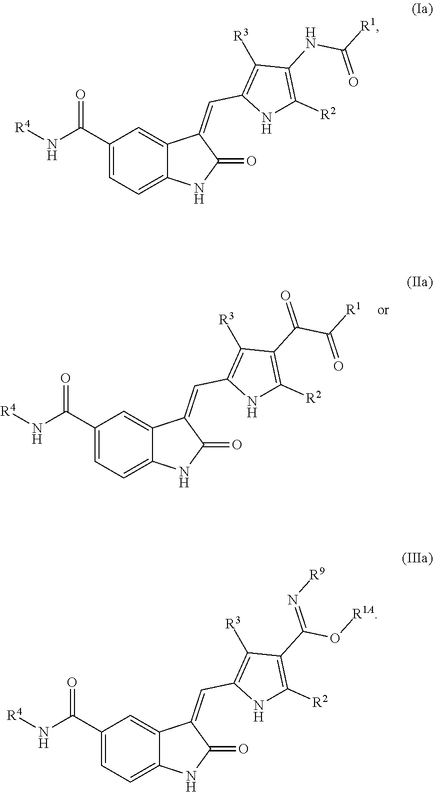

3. The compound of claim 1, or a pharmaceutically acceptable salt thereof, wherein R² and R³ are each, independently, C₁-C₃-alkyl.

4. The compound of claim 3, or a pharmaceutically acceptable salt thereof, wherein R² and R³ are each methyl.

5. The compound of claim 1, or a pharmaceutically acceptable salt thereof, wherein R⁴ is arylalkyl.

6. The compound of claim 1, or a pharmaceutically acceptable salt thereof, wherein R⁴ is a group of the formula: 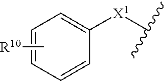
wherein R¹⁰ is H, halo, alkyl, haloalkyl, amido or alkoxy; and
X¹ is absent or alkyl.

7. The compound of claim 6, or a pharmaceutically acceptable salt thereof, wherein R¹⁰ is halo.

8. The compound of claim 6, or a pharmaceutically acceptable salt thereof, wherein R⁴ is a group of the formula: 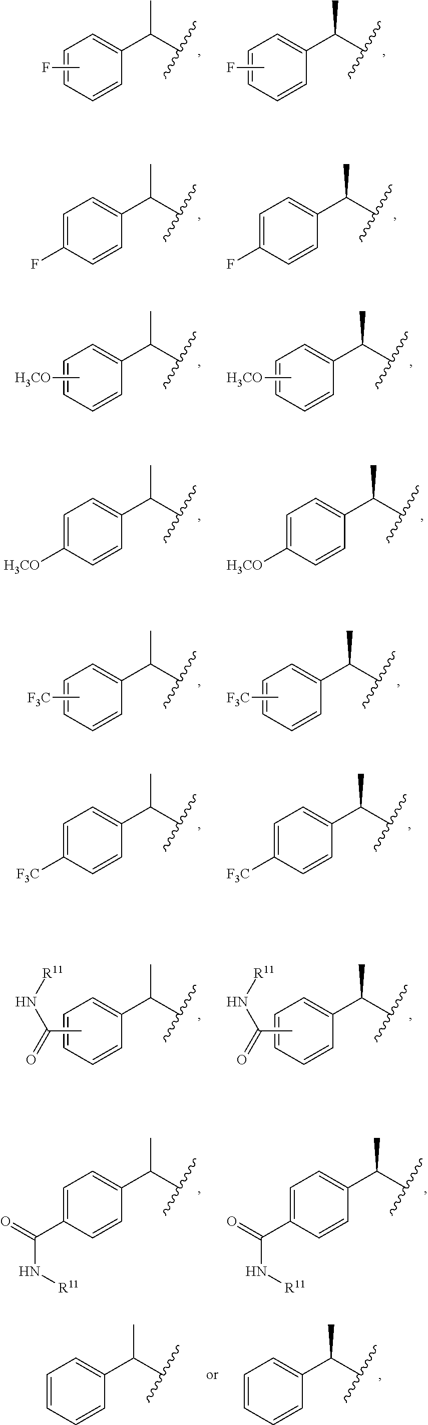
wherein R¹¹ is alkyl.

9. The compound of claim 1, or a pharmaceutically acceptable salt thereof, wherein R⁴ is a group of the formula:

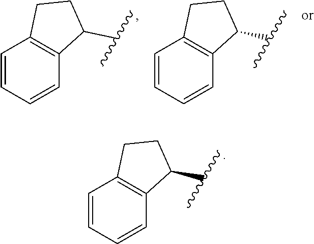

10. The compound of claim 1, or a pharmaceutically acceptable salt thereof, wherein the compound is a compound of the formula: 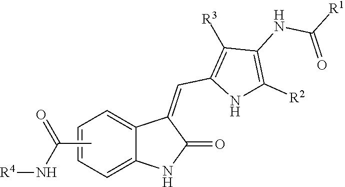
R¹ is R⁵, -alkyl-R⁵, —C(O)R⁵ or 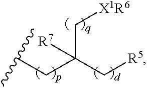
wherein R⁵ is alkyl, cycloalkyl, aryl or heteroaryl.

11. The compound of claim 10, or a pharmaceutically acceptable salt thereof, wherein R⁵ is furanyl, indolyl, imidazolyl, benzimidazolyl or imidazo[1,2-a]pyridinyl; q is 0, p is 0, and d is 0; and/or R¹ is -alkyl-R⁵, such as —(C₁-C₃)alkyl-R⁵.

12. The compound of claim 1, or a pharmaceutically acceptable salt thereof, wherein the compound is a compound of the formula: 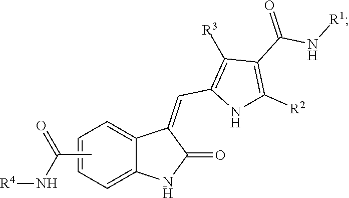
R¹ is R⁵, -alkyl-R⁵ or 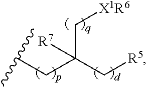
wherein R⁵ is alkyl, hydroxy, alkoxy cycloalkyl, aryl, heterocyclyl or heteroaryl or R⁵ and R⁷ can form a cycloalkyl group.

13. The compound of claim 12, or a pharmaceutically acceptable salt thereof, wherein R⁵ is tetrahydrofuranyl, pyranyl, furanyl, tetrahydro-2H-thiopyran-1,1-dioxide or benzimidazolyl; q is 0 and d is 0; and/or R¹ is -alkyl-R⁵, such as —(C₁-C₃)alkyl-R⁵.

14. The compound of claim 12, or a pharmaceutically acceptable salt thereof, wherein R¹ is 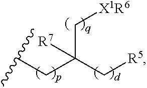
wherein R⁵ and R⁷ form a cycloalkyl group.

15. The compound of claim 12, or a pharmaceutically acceptable salt thereof, wherein R¹ is R⁵ or -alkyl-R⁵ and R⁵ is hydroxy or alkoxy.

16. The compound of claim 1, or a pharmaceutically acceptable salt thereof, wherein the compound is a compound of the formula:

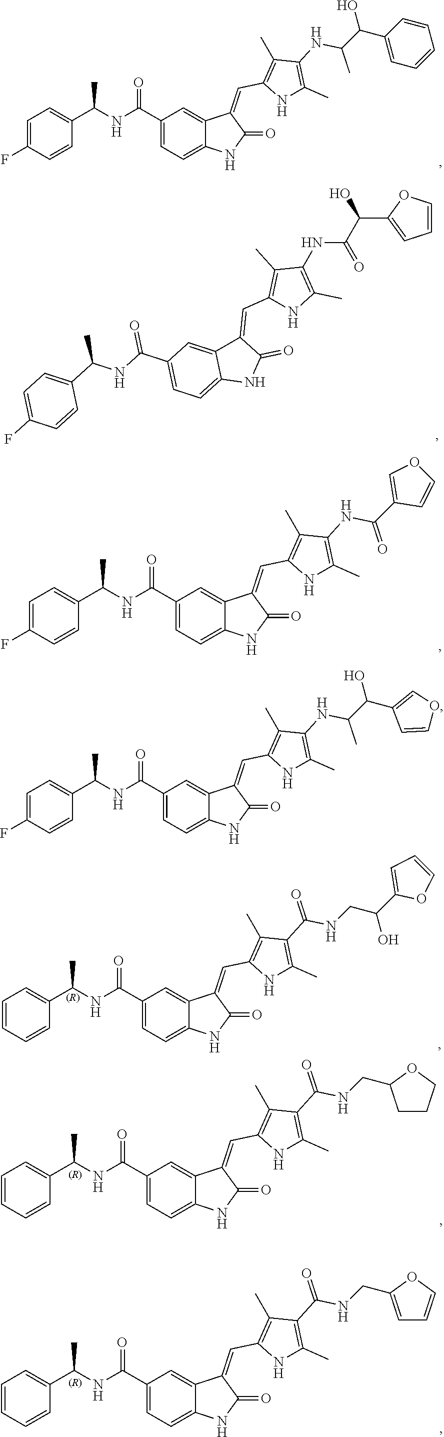

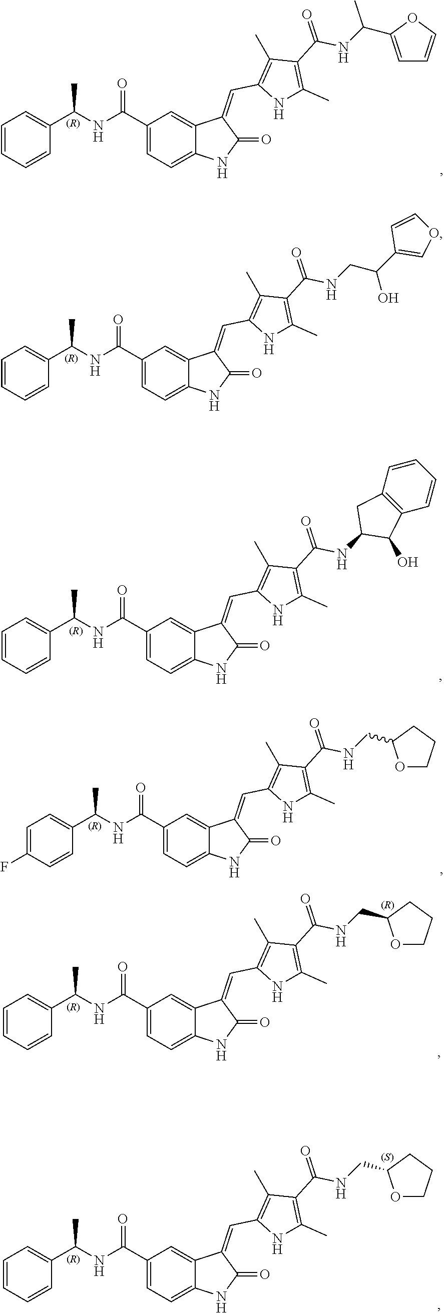

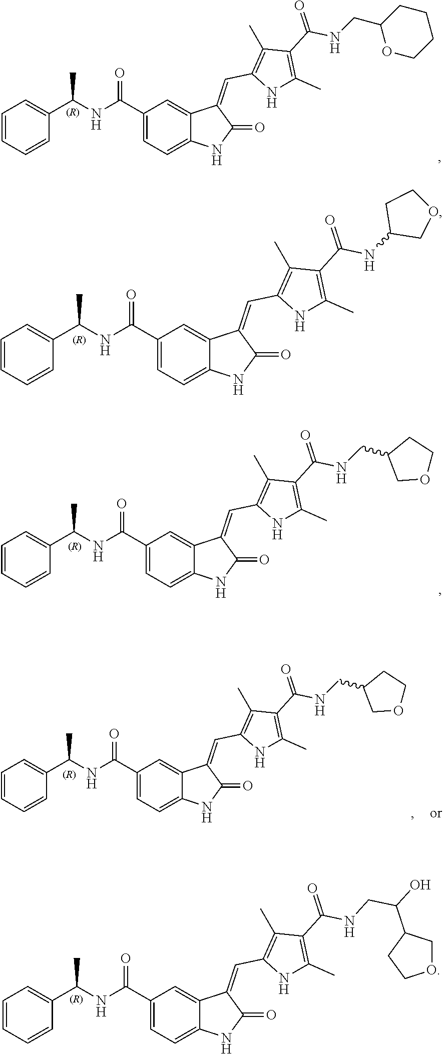

17. The compound of claim 1, or a pharmaceutically acceptable salt thereof, wherein the compound is a compound of the formula:

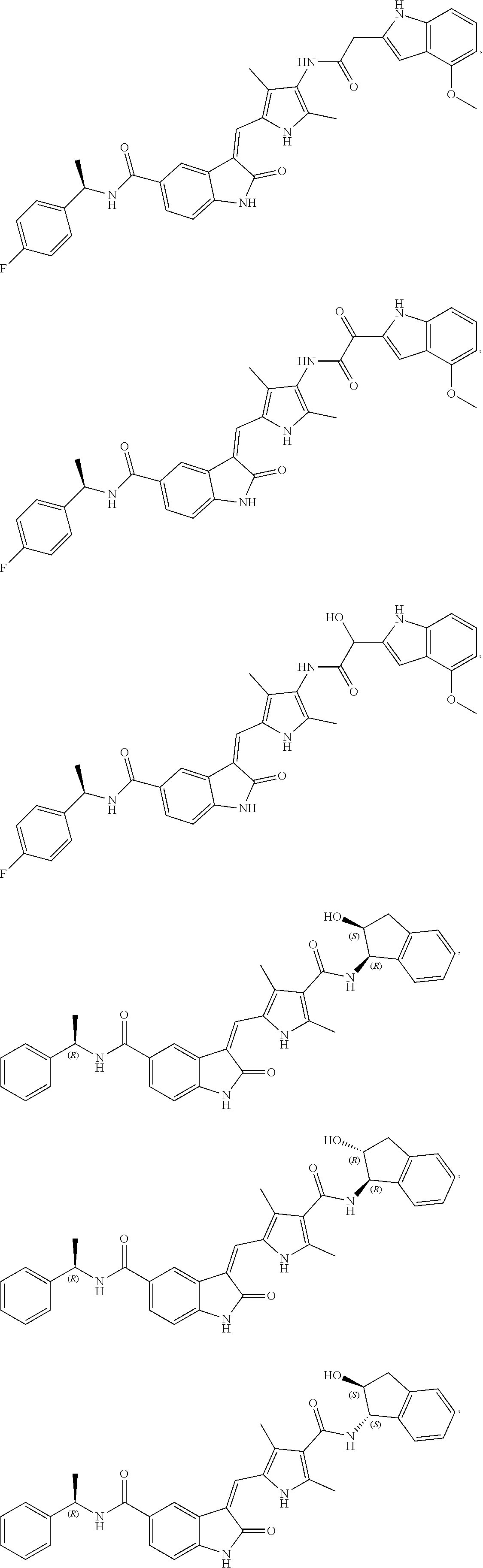

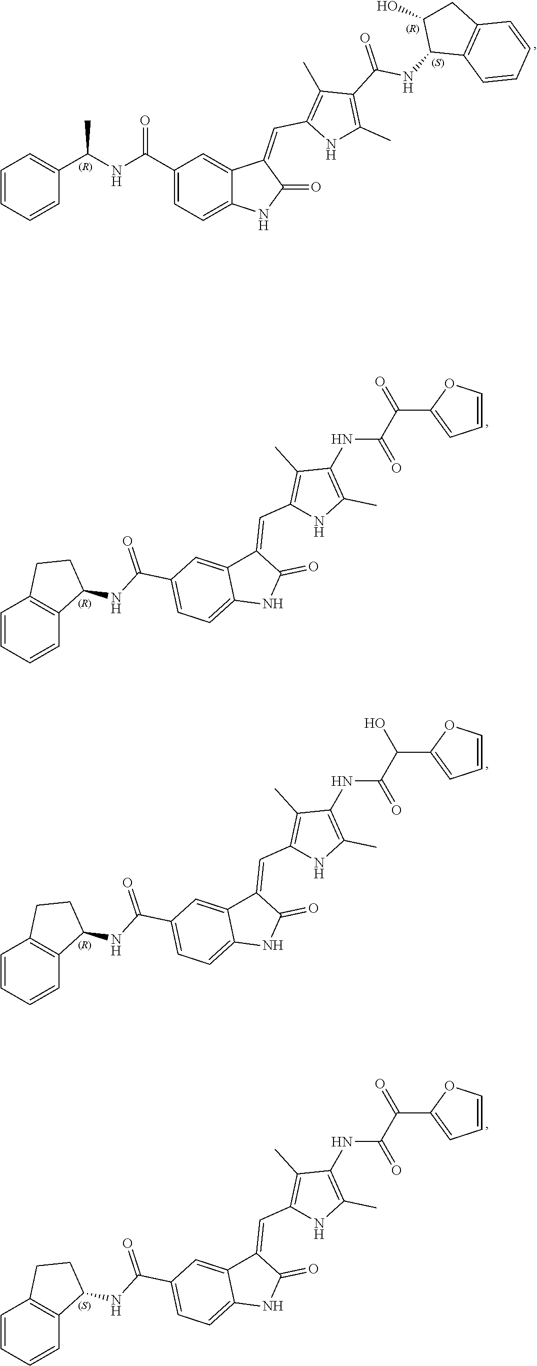

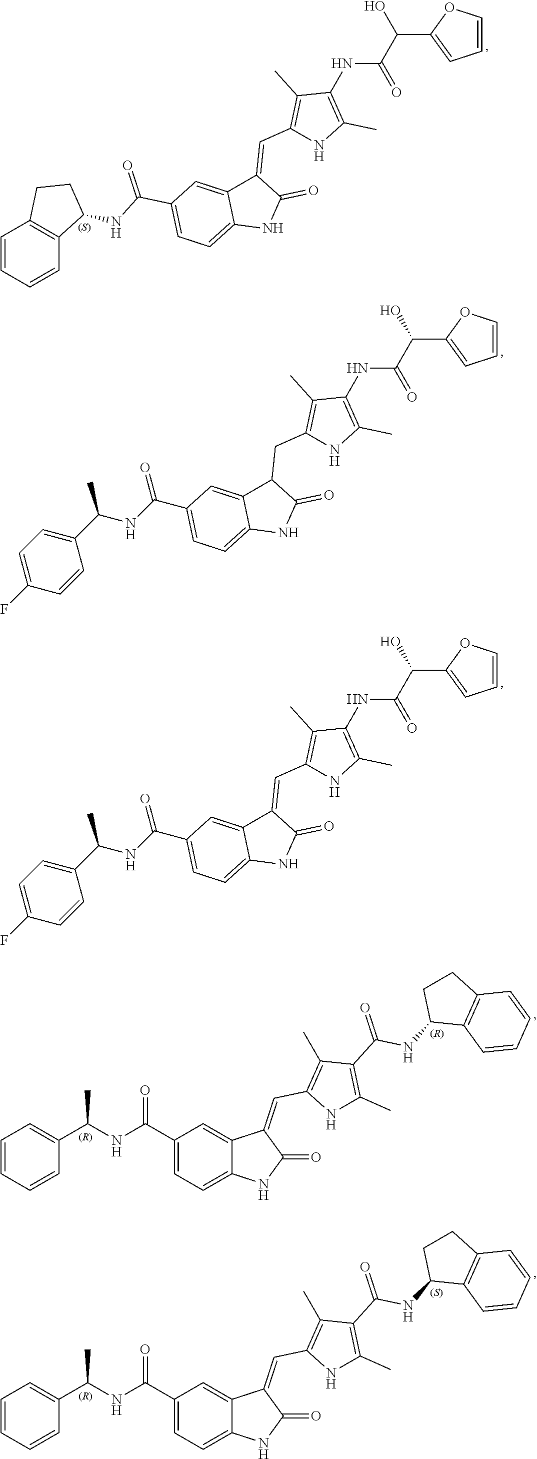

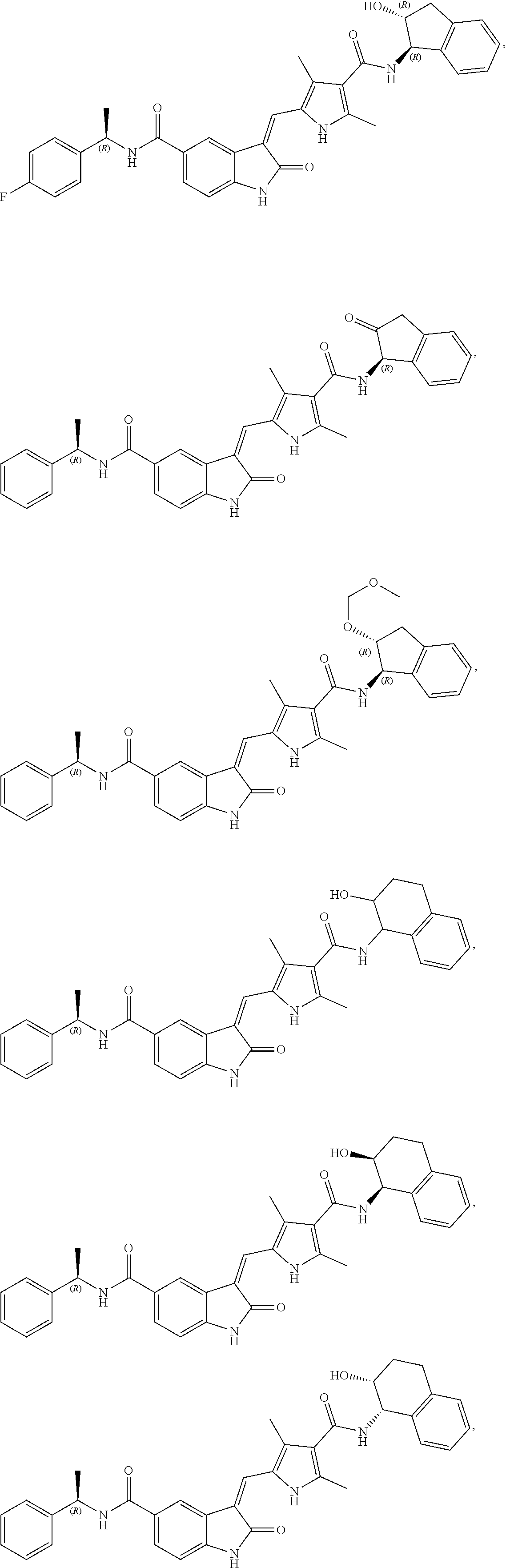

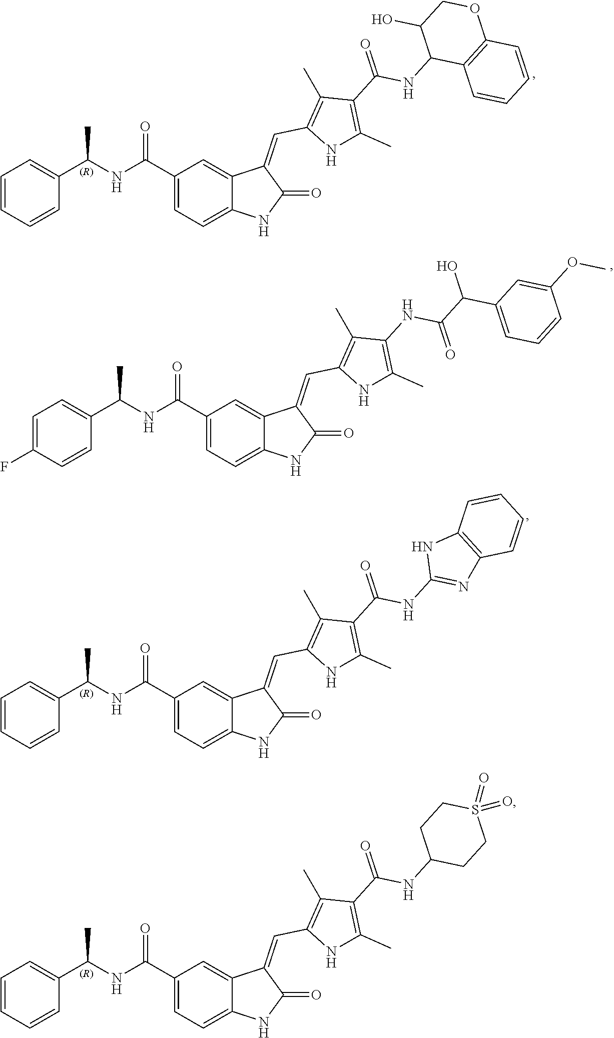

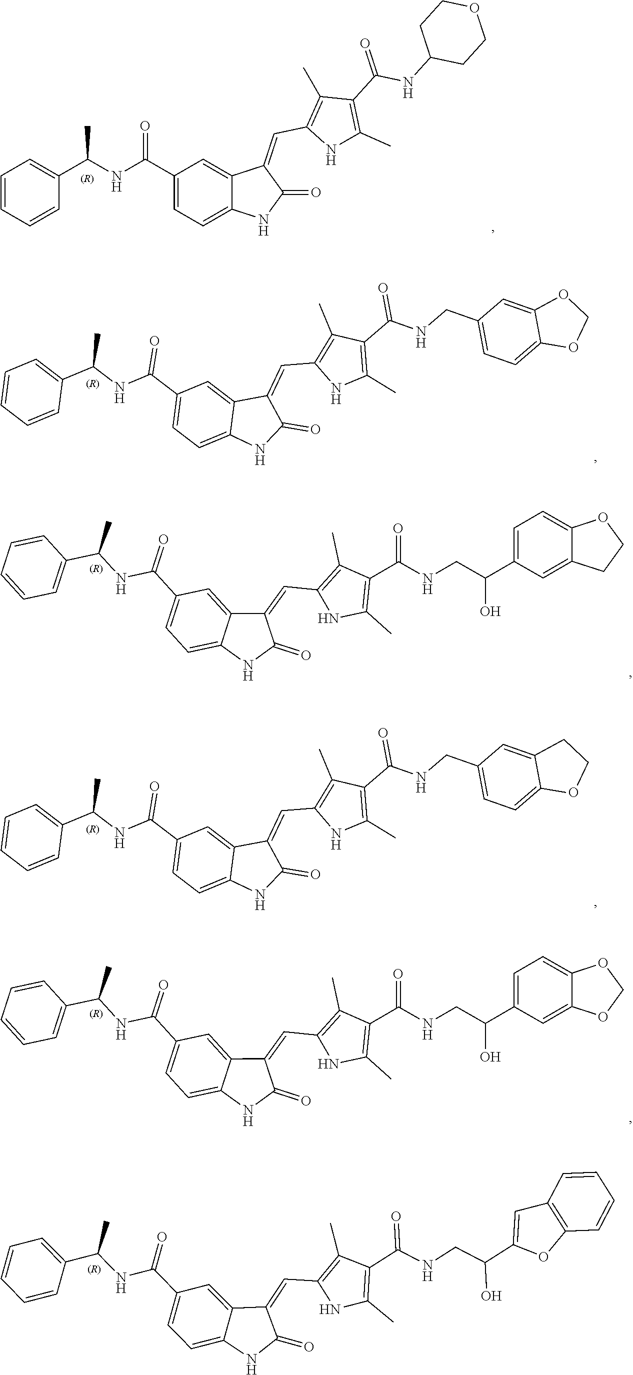

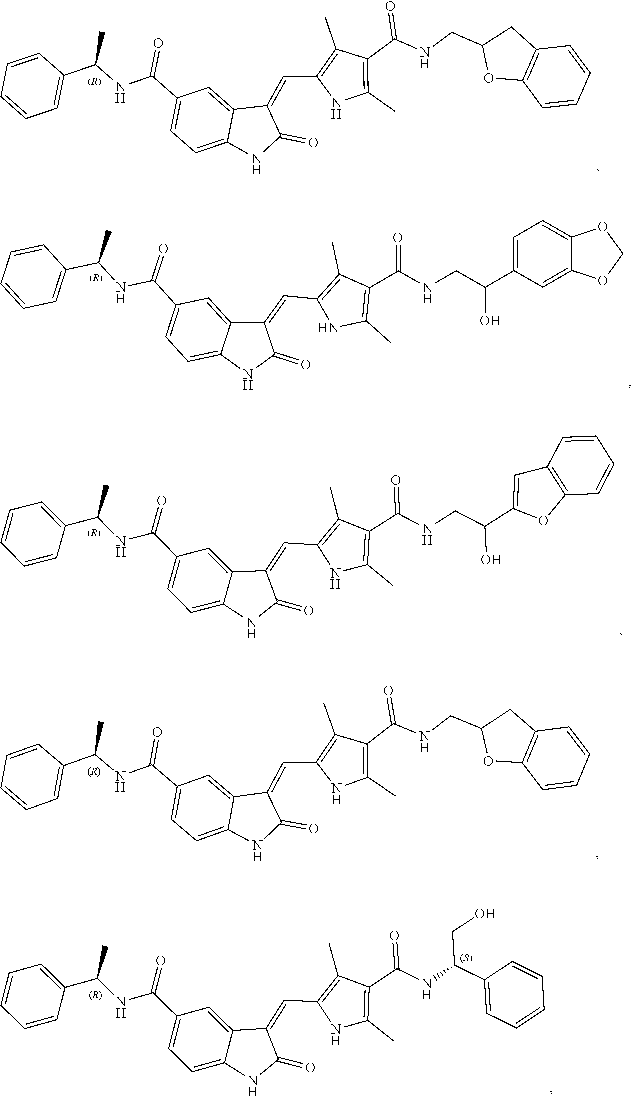

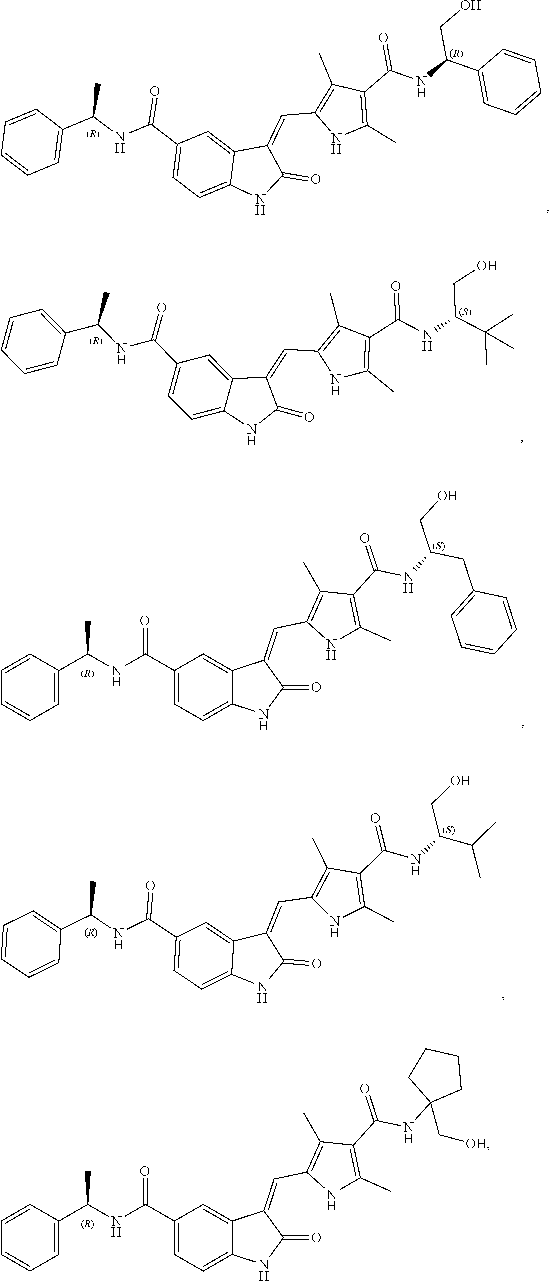

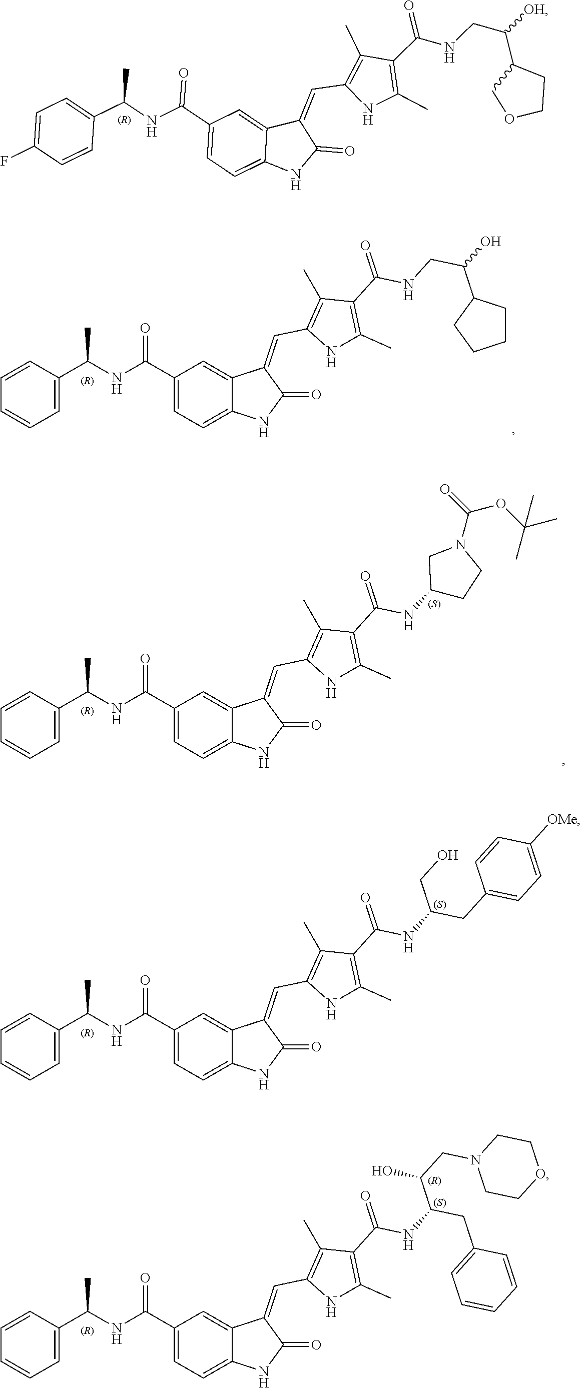

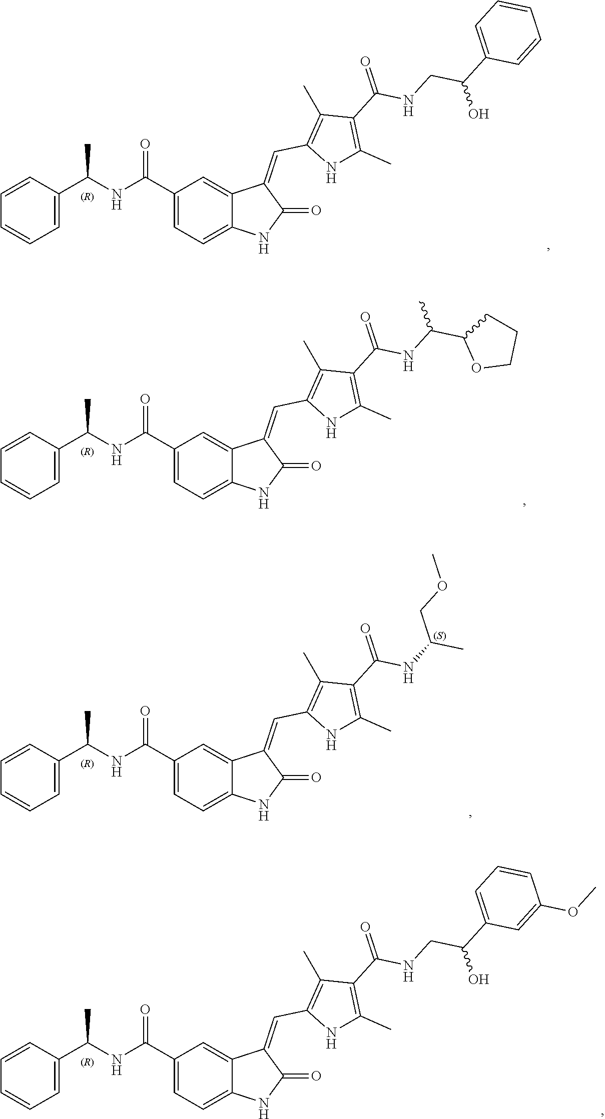

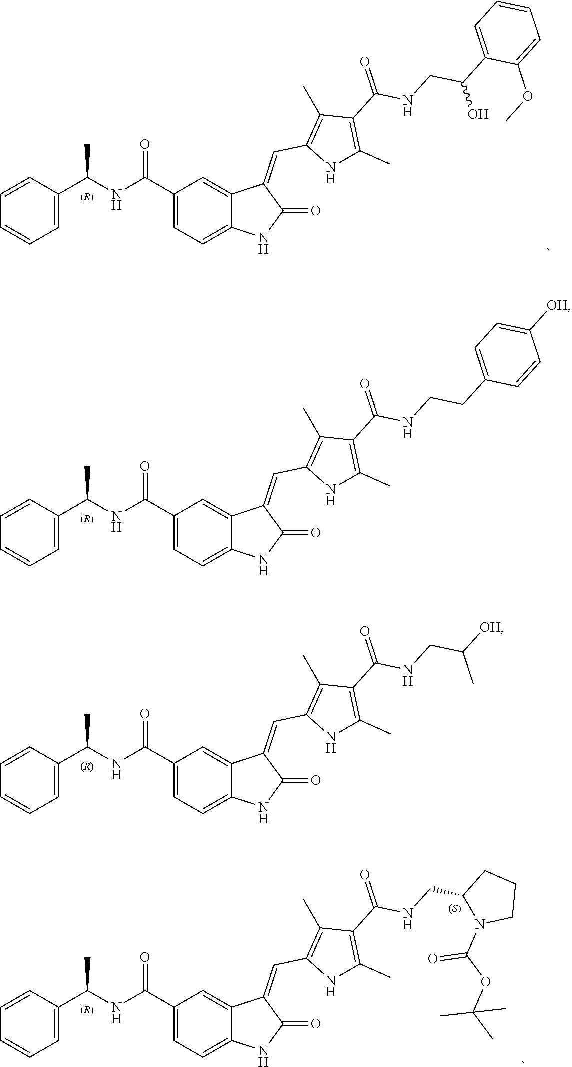

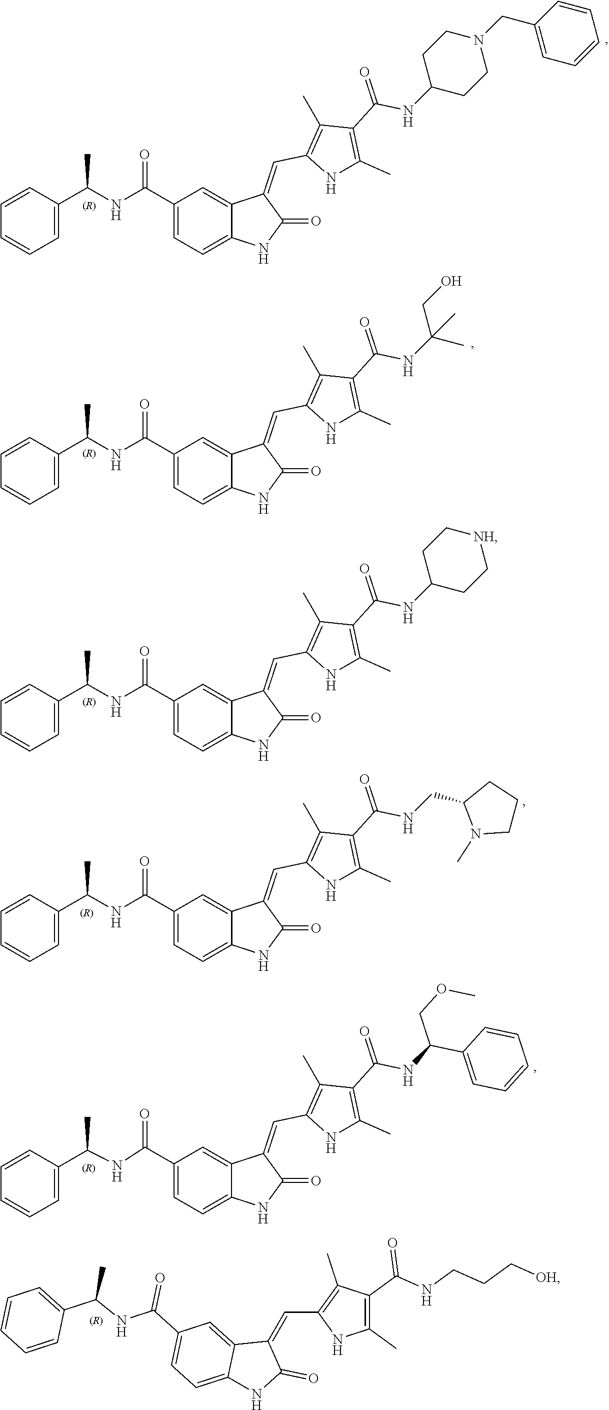

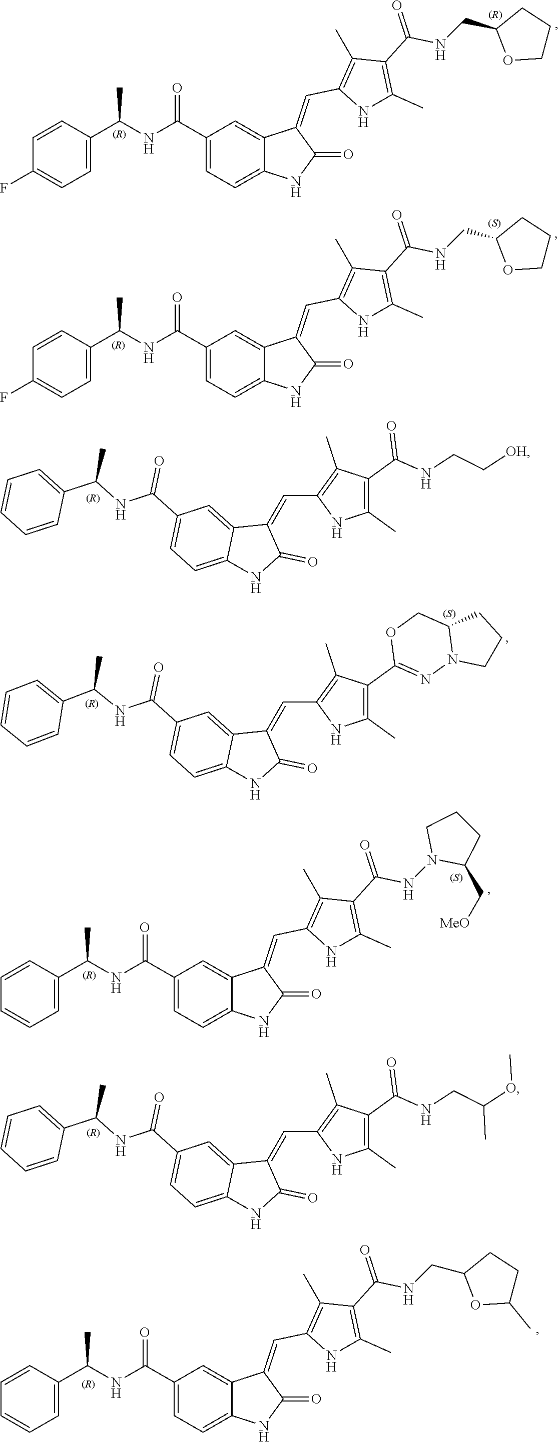

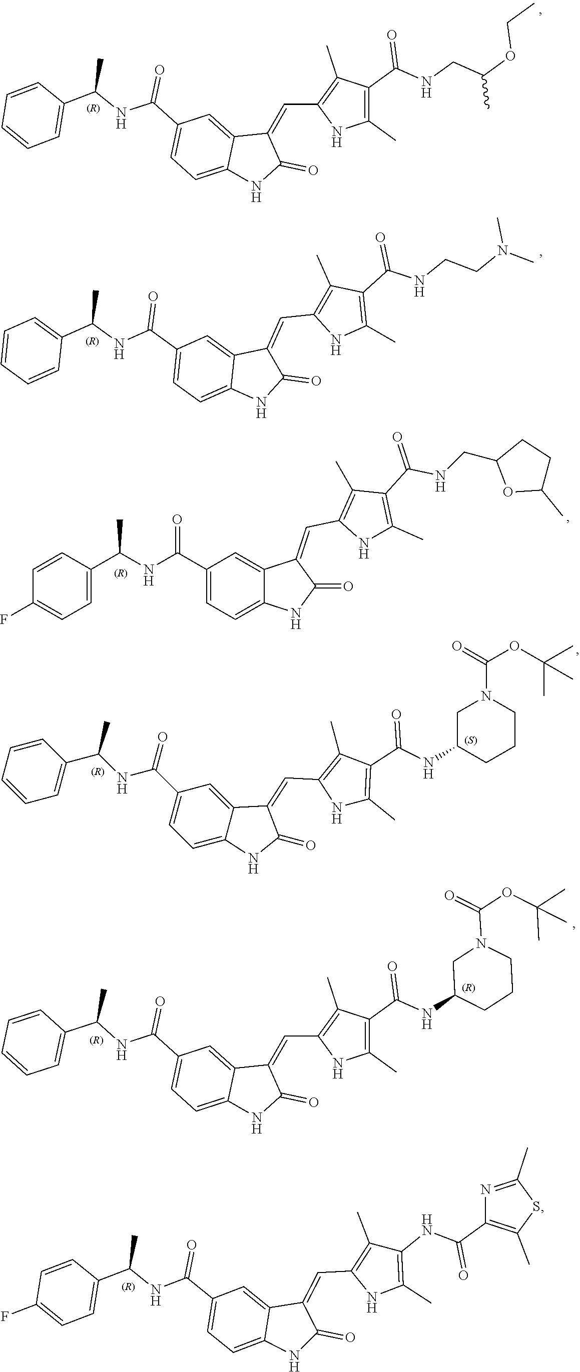

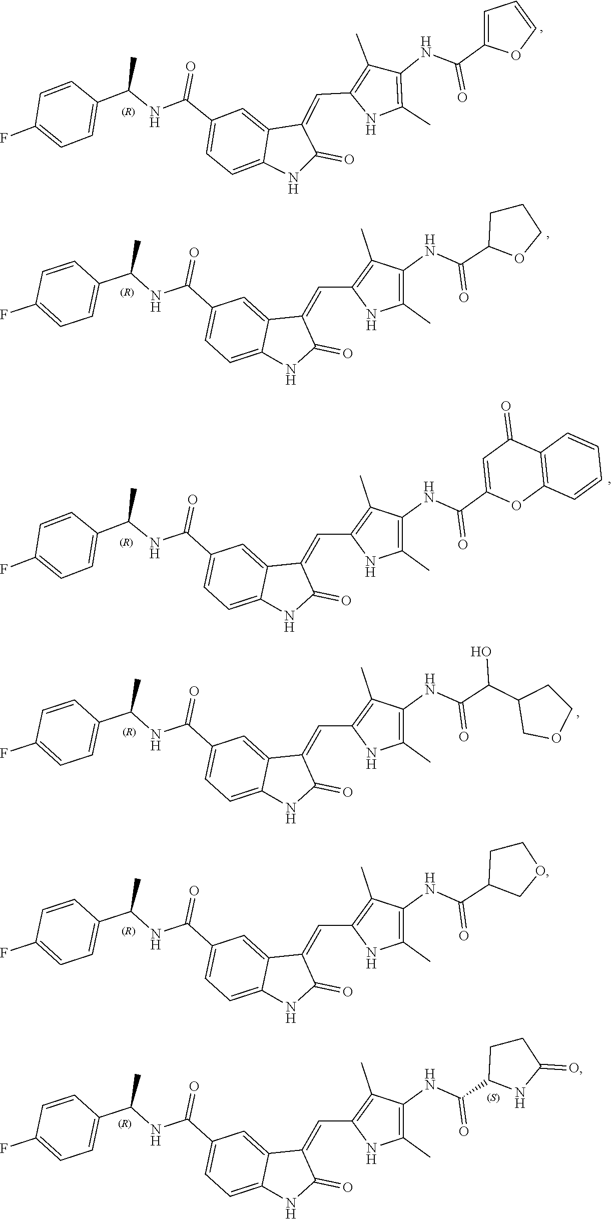

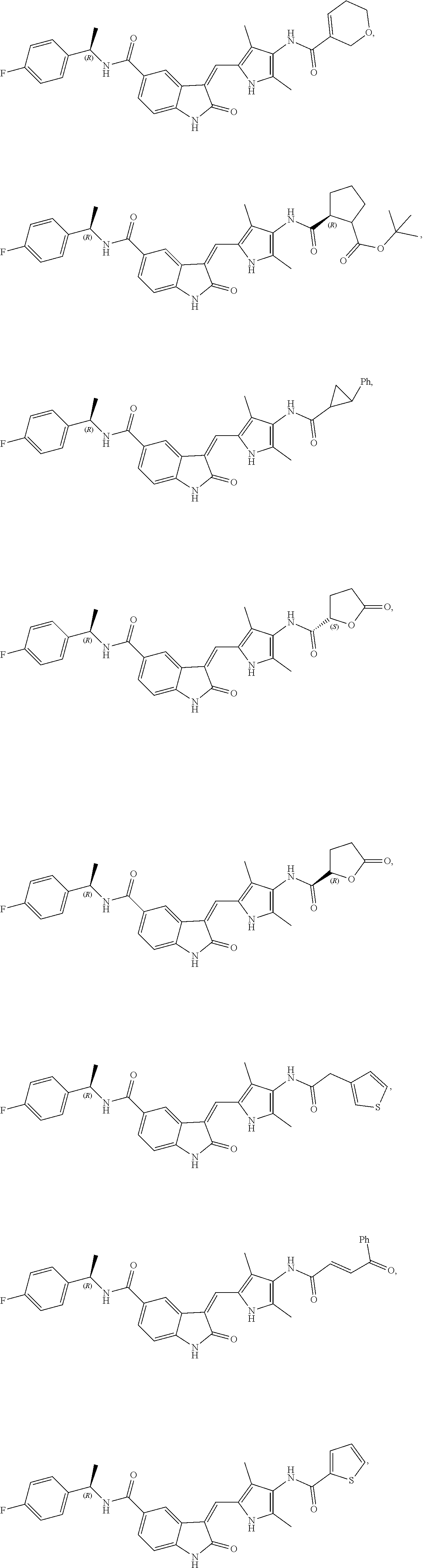

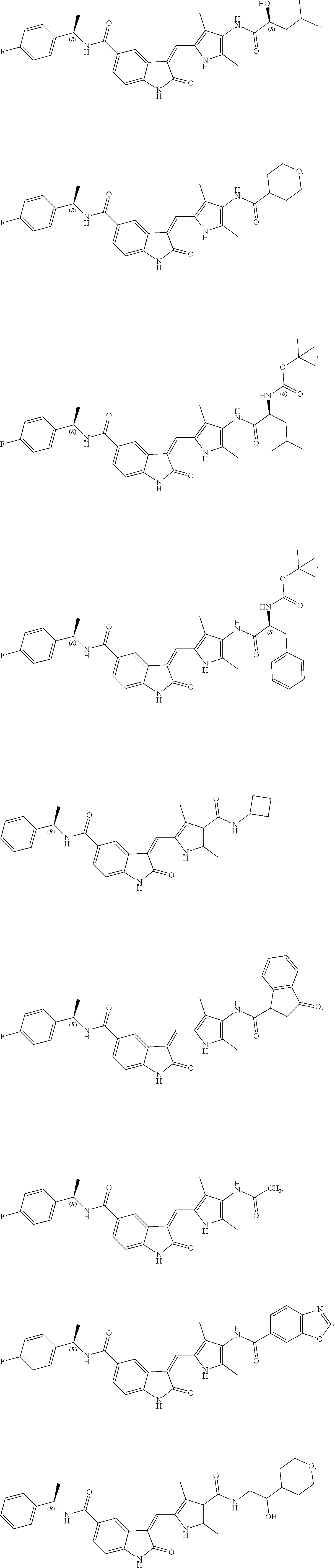

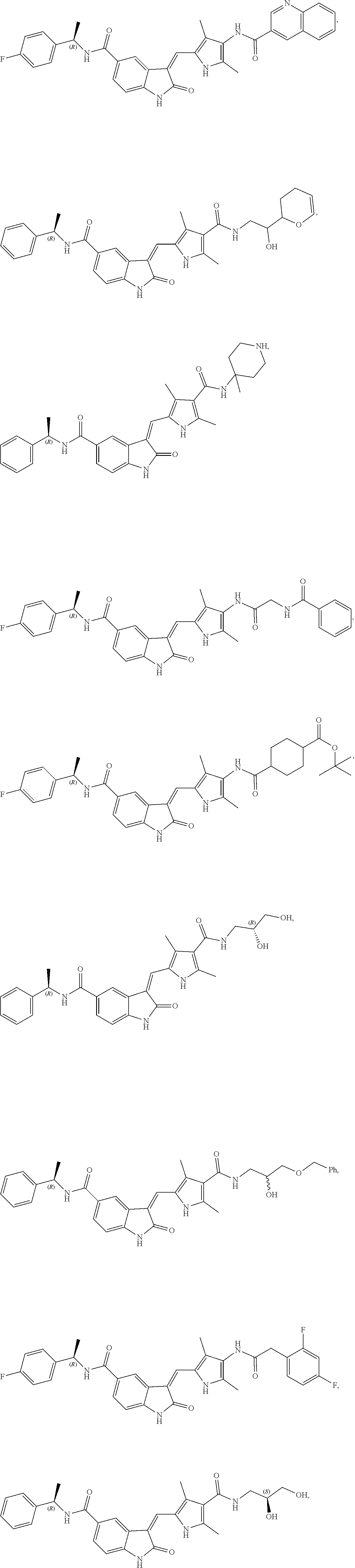

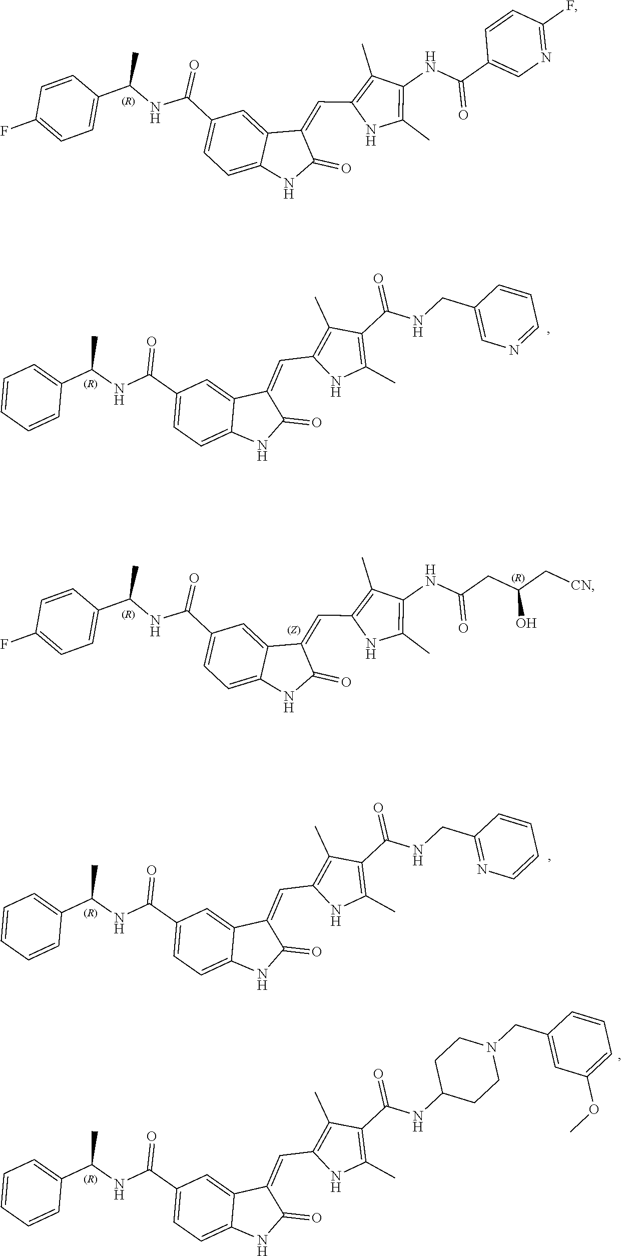

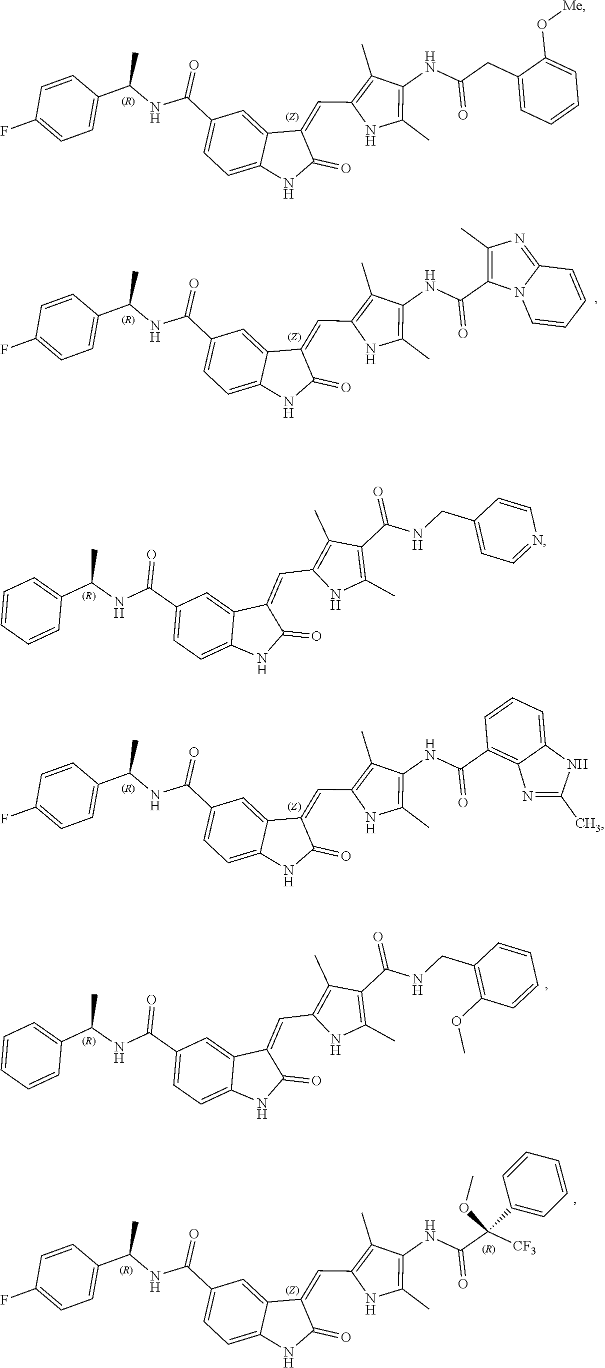

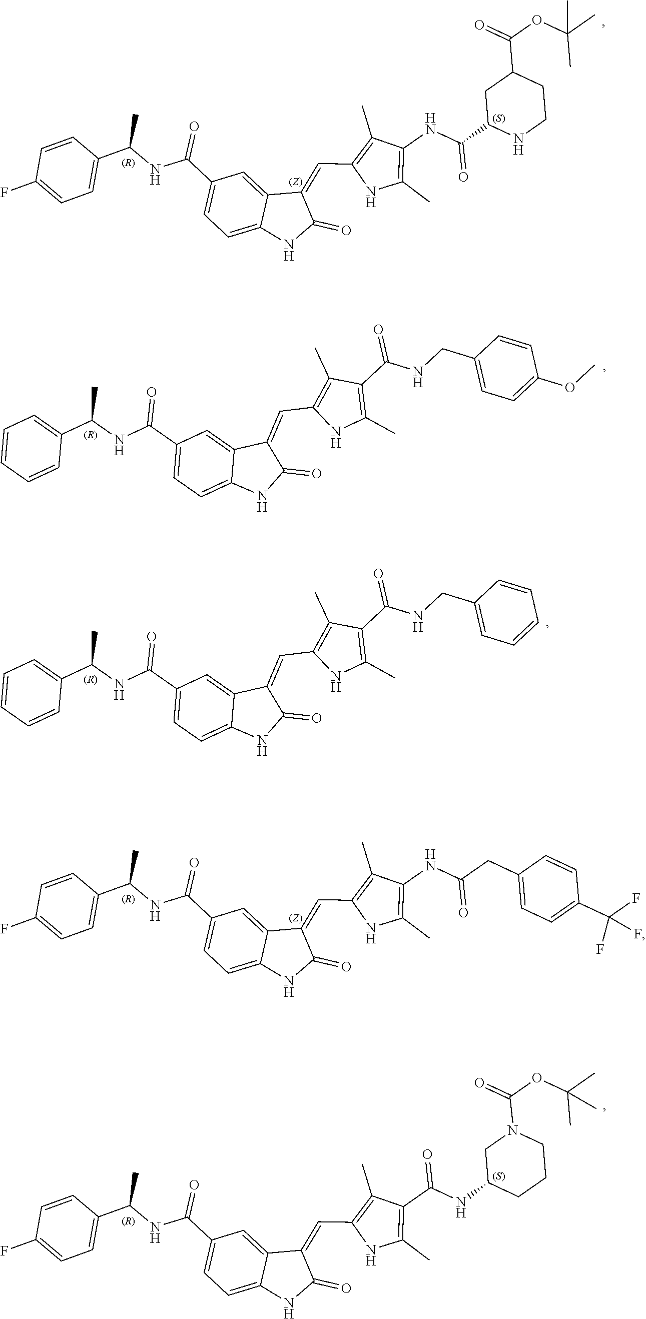

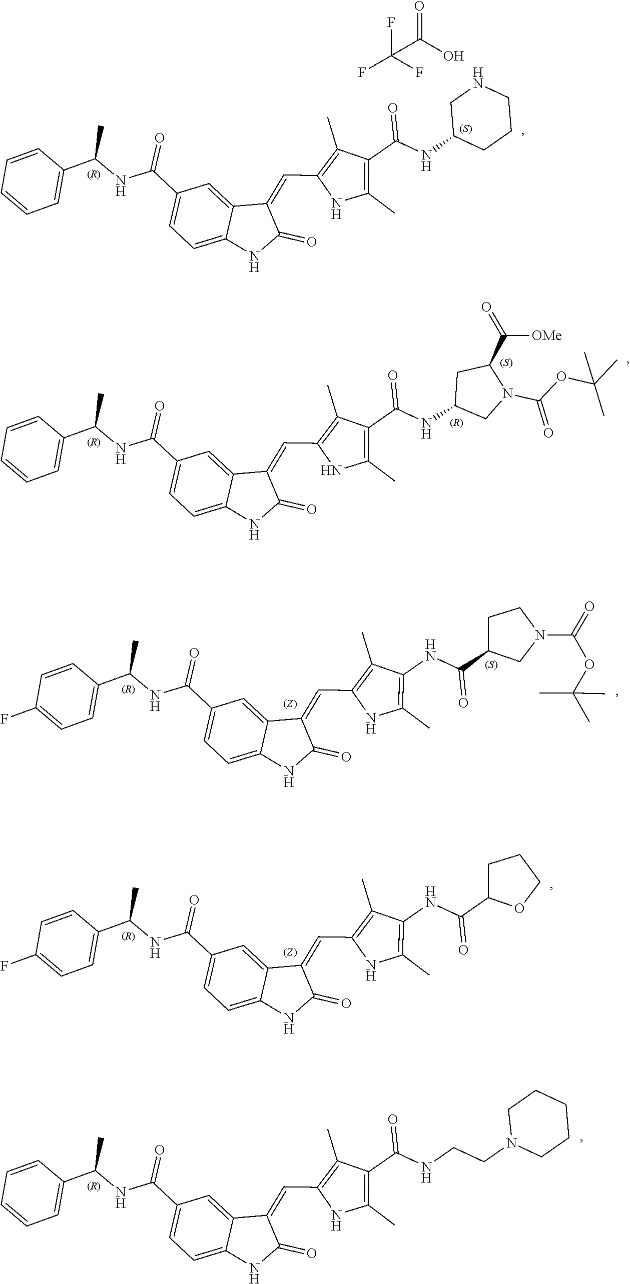

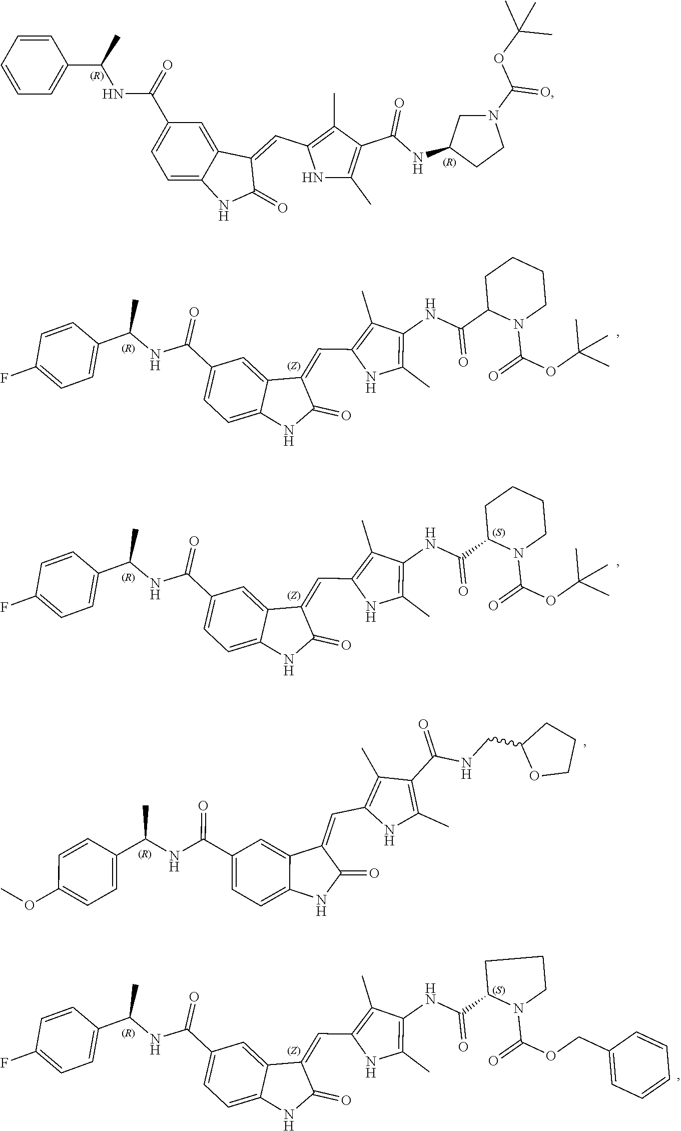

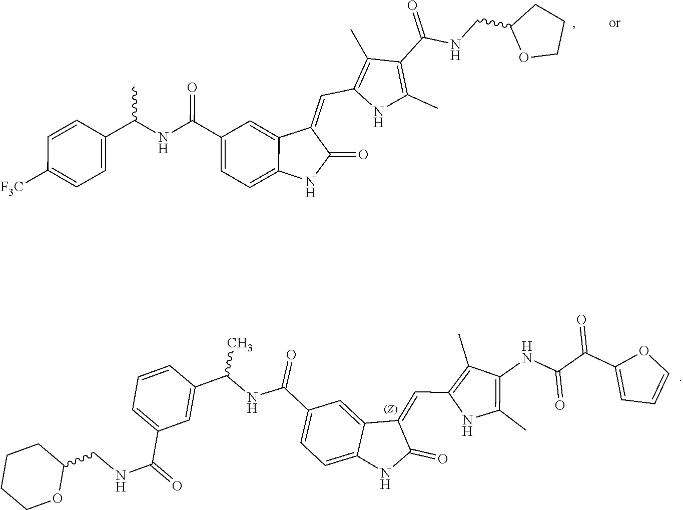

18. A pharmaceutical composition comprising a compound of claim 1, or a pharmaceutically acceptable salt thereof, and at least one pharmaceutically acceptable carrier or excipient.

19. A method of treating a subject in need of inhibition of G protein-coupled receptor kinase 5 (GRK5), which method comprises administering to the subject in need thereof an effective amount of a compound of claim 1, whereupon the subject in need of inhibition of GRK5 is treated.

20. The method of claim 19, wherein the subject has heart disease.

21. The method of claim 20, wherein the heart disease is hypertrophic cardiomyopathy or heart failure.

22. The method of claim 19, wherein the subject has cancer.
